SEQUENCE LISTING (1 sequence)

SEQ ID NO: 1 - DNA, 11 bp, Artificial Sequence
  Description of Artificial Sequence Synthetic oligonucleotide
cttcccttctt

(19) United States
(12) Patent Application Publication
(10) Pub. No.: US 2022/0257599 A1
(43) Pub. Date: Aug. 18, 2022

(54) METHODS USEFUL IN TREATING CANCERS HARBORING A KRAS OR HRAS MUTATION OR AMPLIFICATION

(71) Applicant: Memorial Sloan Kettering Cancer Center, New York, NY (US)

(72) Inventors: Daniel HELLER, New York, NY (US); Christopher HOROSZKO, New York, NY (US)

(21) Appl. No.: 17/626,370

(22) PCT Filed: Jul. 15, 2020
(86) PCT No.: PCT/US2020/042162

§ 371 Date: Jan. 11, 2022

Related U.S. Application Data
(60) Provisional application No. 62/874,474, filed on Jul. 15, 2019.

(51) Int. Cl. A61K 31/517; A61K 31/4709; A61K 45/06; A61P 35/00

(52) U.S. Cl. CPC A61K 31/517; A61P 35/00; A61K 45/06; A61K 31/4709

(57) ABSTRACT

The present technology is directed to methods useful in treating cancer, slowing growth of a tumor, reversing growth of a tumor, slowing growth of a neoplasm, reversing growth of a neoplasm, slowing proliferation of a neoplasm, and/or reversing proliferation of a neoplasm, for cancers, tumors, and neoplasms harboring a constitutively active variant of one or both of KRAS or HRAS, where the constitutively active variant is a gain of function mutation, a duplication, or a gene amplification.

### CROSS-REFERENCE TO RELATED APPLICATIONS

This application claims the benefit of and priority to U.S. Provisional Appl. No. 62/874,474, filed Jul. 15, 2019, which in incorporated herein by reference in its entirety.

### U.S. GOVERNMENT RIGHTS

This invention was made with government support under HD075698 awarded by the National Institutes of Health, and 1752506 awarded by the National Science Foundation. The government has certain rights in the invention.

### FIELD

The present technology is directed to methods useful in treating cancer, slowing growth of a tumor, reversing growth of a tumor, slowing growth of a neoplasm, reversing growth of a neoplasm, slowing proliferation of a neoplasm, and/or reversing proliferation of a neoplasm, for cancers, tumors, and neoplasms harboring a constitutively active variant of one or both of KRAS or HRAS, where the constitutively active variant is a gain of function mutation, a duplication, or a gene amplification.

### SUMMARY

In an aspect, the present technology provides a method of treating a cancer in a subject, where the method includes administering to the subject an effective amount of a compound to treat the cancer; where the compound is at least one of darapladib, rilapladib, AA39-2, or ML256; and where the cancer harbors a constitutively active variant of one or both of KRAS or HRAS, wherein the constitutively active variant is a gain of function mutation, a duplication, or a gene amplification. In any embodiment herein, it may be the cancer exhibits elevated expression and/or activity of at least one of PLA2G7A and PAFAH2 compared to healthy control cells.

In an aspect, the present technology provides a method of slowing or reversing growth of a tumor in a subject, the method comprising administering to the subject an effective amount of a compound; where the compound is at least one of darapladib, rilapladib, AA39-2, or ML256; wherein the effective amount is an amount effective to slow or reverse growth of the tumor; and wherein the tumor is of a cancer that harbors a constitutively active variant of one or both of KRAS or HRAS, wherein the constitutively active variant is a gain of function mutation, a duplication, or a gene amplification. In any embodiment herein, it may be the cancer exhibits elevated expression and/or activity of at least one of PLA2G7A and PAFAH2 compared to healthy control cells.

In an aspect, the present technology provides a method of slowing or reversing growth of a neoplasm in a subject and/or slowing or reversing proliferation of the neoplasm in the subject, the method comprising administering to the subject an effective amount of a compound; where the compound is at least one of darapladib, rilapladib, AA39-2, or ML256; wherein the effective amount is an amount effective to slow or reverse growth of the neoplasm and/or slow or reverse proliferation of the neoplasm; and wherein the neoplasm is of a cancer that harbors a constitutively active variant of one or both of KRAS or HRAS, wherein the constitutively active variant is a gain of function mutation, a duplication, or a gene amplification. In any embodiment herein, it may be the cancer exhibits elevated expression and/or activity of at least one of PLA2G7A and PAFAH2 compared to healthy control cells.

### BRIEF DESCRIPTION OF THE DRAWINGS

FIG. 1 illustrates RAS-induced damage in immortalized mouse embryo fibroblasts, according to the working examples, plotting the change in attached cell number over time versus initial seeding density in the presence of the indicated concentrations of doxycycline. Error bars represent standard deviation (n=3).

FIG. 2 provides immunoblots of vector/iKRas cells 24 h after introducing doxycycline, according to the working examples. Two-tailed t-test (n=3): pixel density of doxycycline paired iKRas and vector. TBP loading control or phospho/total ratio was used to normalize.

FIG. 3 provides a 72 h immunoblot of vector/iKRas for cell-cycle substrates, according to the working examples. Two-tailed t-test (n=3); pixel intensity of doxycycline paired MEF. TBP was used to normalize.

FIG. 4 provides a quantification of integrated intensities from immunofluorescence images containing γH2A.X positive signal, according to the working examples. Error is mean±SEM (One-way ANOVA, n=3, 10 imaging fields at 20× each).

FIG. 5 provides a 24 h and a 72 h immunoblot of vector/iKRas for JNK (top) or ATF-2 (bottom), according to the working examples. Two-tailed t-test (n=3); pixel intensity of doxycycline paired MEF. Phospho/total ratio was used to normalize.

FIG. 6 provides a SA-β-Galactosidase assay (left, scale bar=30 μm), and indirect immunofluorescence for HP1γ nuclear foci (right, scale bar=10 μm), according to the working examples. β-Gal color images were deconvolved and analyzed to give % positive mean±standard deviation, denoted for each condition (n=3×10 imaging fields at 10× magnification). % positive SAHF was calculated using 5+ foci nuclei/total nuclei (mean±standard deviation denoted for each condition, n=3×10 imaging fields at 40× magnification).

FIG. 7 provides a Pan-sPLA2 activity assay of cell lysate or conditioned media using a diheptanoyl thio-PC substrate and DNTB chromogen, according to the working examples. Error bars represent standard deviation (n=3).

FIG. 8 provides a Pan-PAF-AH activity assay of cell lysate or conditioned media using a thio-PAF substrate and DNTB chromogen, according to the working examples. All samples were spin-filtered using a 10 kDa membrane. Error bars represent standard deviation (Two-tailed t-test, n=3). LOD: limit of detection.

FIG. 9 provides the results of confocal immunofluorescence imaging of iKRas or vector stained with PLA2G7A antibody (red secondary), according to the working examples. Blue=Hoechst nuclear stain. Scale bars=10 μm.

FIG. 10 provides quantification of sensor emission wavelengths for the indicated immortalized MEF lines, according to the working examples. Error bars represent standard deviation (Two-tailed t-test, n=3×12-15 imaging fields).

FIG. 11 illustrates lipid reporter response in cells treated with pathway inhibitors or a PAF-AH inducer, according to the working examples, providing reporter emission wavelength from 24 h treated cultures. Error is mean±SD (One-way ANOVA vs. vehicle, n=3×12-15 imaging fields). A schematic of each inhibitor's reported target is included.

FIG. 12 illustrates lipid reporter response in cells treated with pathway inhibitors or a PAF-AH inducer, according to the working examples, providing reporter emission wavelength in Atg5 double knock-out SV40LT MEF with or without transduced K-rasG12V. In a separate experiment, cells were exposed to a protease inhibitor cocktail for 5 h (20 μM leupeptin, 20 μM pepstatin, 10 μM E-64). Error is mean±SD (Two-tail t-test, n=3×12-15 imaging fields).

FIG. 13 provides the results of an intracellular endomembrane reporter assay for lysophospholipids, according to the working examples, where emission center wavelength from sensor in the inducible amplified iKRas line is compared to the single-allele knock-in line, eKRas.

FIG. 14 illustrates lipid reporter response in cells treated with pathway inhibitors or a PAF-AH inducer, according to the working examples, providing reporter emission wavelength in cultures treated for 24 h with 100 nM dexamethasone salt. Error is mean±SD (Two-tail t-test, n=3×12-15 imaging fields).

FIG. 15 provides the results of an intracellular endomembrane reporter assay for lysophospholipids, according to the working examples, where illustrated is the quantification of sensor emission wavelengths from vector/iKRas cells incubated with the indicated drugs during the 24 h induction period: 20 nM varespladib, MJ-33; 75 nM BEL; 500 nM MAFP; 50 nM darapladib, rilapladib, ML256, AA39-2; 800 nM P11; 1 μM TSI-01. Error bars represent standard deviation (Two-tailed t-test vs. vehicle or indicated by brackets; one-way ANOVA vs. vehicle, n=3×12-15 imaging fields).

FIG. 16 provides the results of an intracellular endomembrane reporter assay for lysophospholipids, according to the working examples, where illustrated are sensor emission center wavelength from vector/iKRas cells incubated with 20 μM±-α-tocopherol during the 24 h induction period. Error bars represent standard deviation (Two-tailed t-test, n=3×12-15 imaging fields).

FIG. 17 provides quantification of electron micrographs (n=25 fields) from 12-25,000× imaging of iKRas. Error is mean±SD (one-way ANOVA vs. vector).

FIG. 18 provides the total lipid hydroperoxides of vector control and iKRas cell lysates, via quantification of thiocynate ion chromogen at 500 nm. Error bars represent standard deviation (Two-tailed t-test, n=3). LOD: limit of detection.

FIG. 19 provides a similar quantification as in FIG. 18 but for iKRas cells treated with vehicle or darapladib (50 nM).

FIG. 20 illustrates the effect of group 7 sPLA2 substrate and product lipids on cell proliferation, according to the working examples, providing the change in attached cell number as a function of lipid concentrations spiked into culture medium. Error bars represent standard deviation (n=3).

FIG. 21 provides similarly as FIG. 20 but for the iKRas or eKRas cell lines exposed to 10 μM of each lipid.

FIG. 22 provides immunoblots of vector/iKRas after 24 h exposure to 10 μM lysoPC or cPAF lipids. Two-tailed t-test (n=3): pixel density of paired iKRas and vector treatment conditions. TBP loading control or phospho/total ratio was used to normalize. PAF-R: platelet activating factor receptor.

FIG. 23 illustrates the effect of group 7 sPLA2 targeting on cell survival, according to the working examples, providing the change in attached cell numbers 96 h after exposure to 45 nM siRNA. Error bars represent standard deviation (one-way ANOVA, n=3).

FIG. 24 illustrates the effect of group 7 sPLA2 targeting on cell survival, according to the working examples, providing the change in LDH activity from independent experiments. Data is shown as mean±SEM (one-way ANOVA, n=3).

FIG. 25 illustrates the effect of group 7 sPLA2 targeting on cell survival, according to the working examples, providing the viability of several cell lines in response to darapladib. N=3 replicates; error bars represent standard deviation.

FIG. 26 illustrates the effect of darapladib on eKRas and wildtype (WT) MEF, according to the working examples. Cells were plated as described in the main text for viability testing. Darapladib was titrated into cell culture at concentrations above its in vitro EC₅₀ (10-1000 nM). N=3 replicates, error±SD; data fit with sigmoidal dose-response.

FIG. 27 illustrates the effect of group 7 sPLA2 targeting on cell survival, according to the working examples, providing the results of a survival study of mice injected with KP lung cells on Day 0 and treated with vehicle or darapladip (Darap, 10 mg/kg) daily (oral gavage) starting on Day 1 (D1) or Day 6 (D6). N(vehicle)=8, N(Darap, D1)=8, N(Darap, D6)=9.

### DETAILED DESCRIPTION

The following terms are used throughout as defined below.

As used herein and in the appended claims, singular articles such as “a” and “an” and “the” and similar referents in the context of describing the elements (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the embodiments and does not pose a limitation on the scope of the claims unless otherwise stated. No language in the specification should be construed as indicating any non-claimed element as essential.

As used herein, “about” will be understood by persons of ordinary skill in the art and will vary to some extent depending upon the context in which it is used. If there are uses of the term which are not clear to persons of ordinary skill in the art, given the context in which it is used, “about” will mean up to plus or minus 10% of the particular term—for example, “about 10 wt. %” would be understood to mean “9 wt. % to 11 wt. %.” It is to be understood that when “about” precedes a term, the term is to be construed as disclosing “about” the term as well as the term without modification by “about”—for example, “about 10 wt. %” discloses “9 wt. % to 11 wt. %” as well as disclosing “10 wt. %.”

The phrase “and/or” as used in the present disclosure will be understood to mean any one of the recited members individually or a combination of any two or more thereof—for example, “A, B, and/or C” would mean “A, B, C, A and B, A and C, or B and C.”

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into subranges as discussed above. Finally, as will be understood by one skilled in the art, a range includes each individual member. Thus, for example, a group having 1-3 atoms refers to groups having 1, 2, or 3 atoms. Similarly, a group having 1-5 atoms refers to groups having 1, 2, 3, 4, or 5 atoms, and so forth.

Pharmaceutically acceptable salts of compounds described herein are within the scope of the present technology and include acid or base addition salts which retain the desired pharmacological activity and is not biologically undesirable (e.g., the salt is not unduly toxic, allergenic, or irritating, and is bioavailable). When the compound of the present technology has a basic group, such as, for example, an amino group, pharmaceutically acceptable salts can be formed with inorganic acids (such as hydrochloric acid, hydroboric acid, nitric acid, sulfuric acid, and phosphoric acid), organic acids (e.g. alginate, formic acid, acetic acid, benzoic acid, gluconic acid, fumaric acid, oxalic acid, tartaric acid, lactic acid, maleic acid, citric acid, succinic acid, malic acid, methanesulfonic acid, benzenesulfonic acid, naphthalene sulfonic acid, and p-toluenesulfonic acid) or acidic amino acids (such as aspartic acid and glutamic acid). When the compound of the present technology has an acidic group, such as for example, a carboxylic acid group, it can form salts with metals, such as alkali and earth alkali metals (e.g. Na⁺, Li⁺, K⁺, Ca²⁺, Mg²⁺, Zn²⁺) ammonia or organic amines (e.g. dicyclohexylamine, trimethylamine, triethylamine, pyridine, picoline, ethanolamine, diethanolamine, triethanolamine) or basic amino acids (e.g. arginine, lysine and ornithine). Such salts can be prepared in situ during isolation and purification of the compounds or by separately reacting the purified compound in its free base or free acid form with a suitable acid or base, respectively, and isolating the salt thus formed.

Those of skill in the art will appreciate that compounds of the present technology may exhibit the phenomena of tautomerism, conformational isomerism, geometric isomerism and/or stereoisomerism. As the formula drawings within the specification and claims can represent only one of the possible tautomeric, conformational isomeric, stereochemical or geometric isomeric forms, it should be understood that the present technology encompasses any tautomeric, conformational isomeric, stereochemical and/or geometric isomeric forms of the compounds having one or more of the utilities described herein, as well as mixtures of these various different forms.

“Tautomers” refers to isomeric forms of a compound that are in equilibrium with each other. The presence and concentrations of the isomeric forms will depend on the environment the compound is found in and may be different depending upon, for example, whether the compound is a solid or is in an organic or aqueous solution. For example, in aqueous solution, quinazolinones may exhibit the following isomeric forms, which are referred to as tautomers of each other:

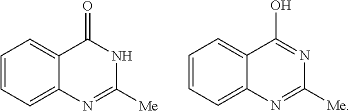 [SMILES: Cc1nc2ccccc2c(=O)[nH]1 ; Cc1nc(O)c2ccccc2n1]

As another example, guanidines may exhibit the following isomeric forms in protic organic solution (e.g., water), also referred to as tautomers of each other:

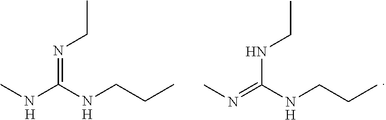

Because of the limits of representing compounds by structural formulas, it is to be understood that all chemical formulas of the compounds described herein represent all tautomeric forms of compounds and are within the scope of the present technology.

Stereoisomers of compounds (also known as optical isomers) include all chiral, diastereomeric, and racemic forms of a structure, unless the specific stereochemistry is expressly indicated. Thus, compounds used in the present technology include enriched or resolved optical isomers at any or all asymmetric atoms as are apparent from the depictions. Both racemic and diastereomeric mixtures, as well as the individual optical isomers can be isolated or synthesized so as to be substantially free of their enantiomeric or diastereomeric partners, and these stereoisomers are all within the scope of the present technology.

The compounds of the present technology may exist as solvates, especially hydrates. Hydrates may form during manufacture of the compounds or compositions comprising the compounds, or hydrates may form over time due to the hygroscopic nature of the compounds. Compounds of the present technology may exist as organic solvates as well, including DMF, ether, and alcohol solvates among others. The identification and preparation of any particular solvate is within the skill of the ordinary artisan of synthetic organic or medicinal chemistry.

Throughout this disclosure, various publications, patents, and published patent specifications are referenced by an identifying citation. Also within this disclosure are Arabic numerals referring to referenced citations, the full bibliographic details of which are provided preceding the claims. The disclosures of these publications, patents, and published patent specifications are hereby incorporated by reference into the present disclosure. Definitions that are contained in text incorporated by reference are excluded to the extent that they contradict definitions in this disclosure.

### The Present Technology

Stress-induced premature senescence is a tumor-suppressive mechanism that delays or aborts oncogenesis. Constitutive signaling through RAS/RAF, important components of the mitogenic MAP kinase cascade, can trigger proliferation delay, apoptosis,^{1, 2, 3, 4, 5, 6, 7, 8} and DNA damage responses⁹ in vitro and in vivo.^{7, 10, 11, 12, 13} While this process may suppress tumor initiation, oncogene-induced senescence (OIS) can be detrimental since surviving cells lose genomic stability through, for example, oxidative damage and replication defects.^{14, 15, 16, 17, 18}. Consistent with this idea, endogenous mutant KRAS expression in the developing mouse embryo causes pre-malignant tissue defects: senescence, p21^{waf1/cip1} overexpression, DNA damage responses, mutant allele instability, and overall developmental delay.^{19, 20, 21, 22} More recently, epithelial cells and fibroblasts have been used to recapitulate oncogene-induced damage; for example, innate inflammatory gene expression phenotypes (senescence inflammatory response, SIR, and senescence-associated secretory phenotype, SASP) are described.^{23, 24} OIS is thought to act as a prompt response to cellular damage by initiating arrest, local inflammation, and extracellular communication. Transcriptome analysis and histology routinely show the expression of genes encoding secretory phospholipase A2 (sPLA2)—conserved inflammatory enzymes—in malignant tissues and cells that harbor the ras oncogene. However, sPLA2 enzymes are classic leukocyte products and their role in cancer biology remains unclear. Interestingly, evidence shows that the products of sPLA2 enzyme activity, lysophospholipids and related species, are generated in abundance during cellular senescence.²⁵ Furthermore, some sPLA2s are directly involved in senescence.^{26, 27, 28, 29} These observations prompted the inventors to evaluate the role of sPLA2s—a large serine hydrolase family, initially studied in animal venom and inflamed joints, that is integral to cardiovascular, lipid, and leukocyte signaling^{30, 31, 32}—within the context of oncogenic RAS-induced damage.

To this end, the inventors investigated the involvement of sPLA2s in a murine model of oncogenic RAS-induced damage. Constitutive KRAS or HRAS was introduced into SV40 mouse embryo fibroblasts (MEF) using a Tet-ON retroviral plasmid (amplification model, iKRas), or through endogenous recombination in mouse embryos (single allele model, eKRas).²³ The inventors compared the extent of cellular damage from oncogenic RAS amplification to the hyperproliferating endogenous mutant. The oncogene amplified line exhibited unique and striking overexpression of p21^{waf1/cip1} doxycycline-dose dependent arrest, senescence-associated markers, DNA damage, and SIR/SASP gene expression. Using enzymatic and lipidomic assays, and a nanosensor of endosomal lipids, the inventors found that a group 7 sPLA2 isoform (group 7 includes pla2g7, PLA2G7A, also known as lipoprotein-associated phospholipase A2 and pafah2, PLA2G7B, also known as platelet-activating factor acetylhydrolase 2) showed activity that was upregulated upon RAS amplification. The inventors found that RAS damage caused lipid hydroperoxidation and an increase in endogenous oxidants, consistent with the specialized oxidized phospholipid clearing role of mobilized group 7 sPLA2 enzymes. Cellular arrest, p21^{waf1/cip1}, and phosphorylated ERK were stimulated on exposure to a non-degradable group 7 substrate lipid. Knockdown of group 7 sPLA2 isoforms selectively killed the RAS mutants. Surprisingly, the inventors found that darapladib, a potent second generation group 7 enzyme inhibitor previously tested in clinical trials for atherosclerosis and Alzheimer's, prevented lysophospholipid accumulation in the RAS-transformed cells, preferentially killed oncogenic ras-harboring lines, and prolonged survival in a Kras^{G12D/+}/p53^{-/-} lung cancer model in mice.

Thus, in an aspect, the present technology provides a method of treating a cancer in a subject, where the method includes administering to the subject an effective amount of a compound to treat the cancer; where the compound is at least one of darapladib, rilapladib, AA39-2, or ML256 (collectively or individually referred to as “a compound of the present technology” or the like; also referred to as “the compound”); and where the cancer harbors a constitutively active variant of one or both of KRAS or HRAS, wherein the constitutively active variant is a gain of function mutation, a duplication, or a gene amplification. The structural formulas for each of darapladib, rilapladib, AA39-2, and ML256 are provided below in Scheme 1.

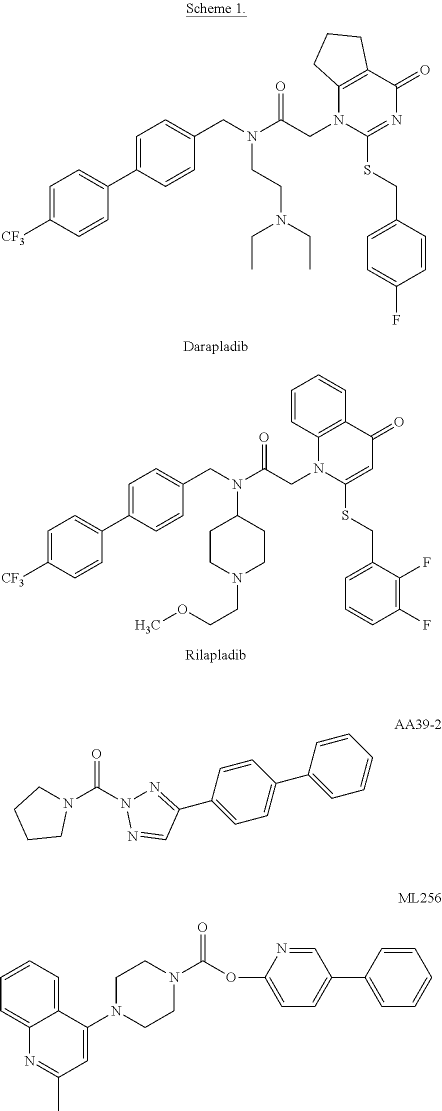 [SMILES: CCN(CC)CCN(Cc1ccc(-c2ccc(C(F)(F)F)cc2)cc1)C(=O)Cn1c(SCc2ccc(F)cc2)nc(=O)c2c1CCC2 ; COCCN1CCC(N(Cc2ccc(-c3ccc(C(F)(F)F)cc3)cc2)C(=O)Cn2c(SCc3cccc(F)c3F)cc(=O)c3ccccc32)CC1 ; O=C(N1CCCC1)n1ncc(-c2ccc(-c3ccccc3)cc2)n1 ; Cc1cc(N2CCN(C(=O)Oc3ccc(-c4ccccc4)cn3)CC2)c2ccccc2n1]

In any embodiment herein, it may be the cancer exhibits elevated expression and/or activity of at least one of PLA2G7A and PAFAH2 compared to healthy control cells.

In an aspect, the present technology provides a method of slowing or reversing growth of a tumor in a subject, the method comprising administering to the subject an effective amount of a compound; where the compound is at least one of darapladib, rilapladib, AA39-2, or ML256; wherein the effective amount is an amount effective to slow or reverse growth of the tumor; and wherein the tumor is of a cancer that harbors a constitutively active variant of one or both of KRAS or HRAS, wherein the constitutively active variant is a gain of function mutation, a duplication, or a gene amplification. In any embodiment herein, it may be the cancer exhibits elevated expression and/or activity of at least one of PLA2G7A and PAFAH2 compared to healthy control cells.

In an aspect, the present technology provides a method of slowing or reversing growth of a neoplasm in a subject and/or slowing or reversing proliferation of the neoplasm in the subject, the method comprising administering to the subject an effective amount of a compound; where the compound is at least one of darapladib, rilapladib, AA39-2, or ML256; wherein the effective amount is an amount effective to slow or reverse growth of the neoplasm and/or slow or reverse proliferation of the neoplasm; and wherein the neoplasm is of a cancer that harbors a constitutively active variant of one or both of KRAS or HRAS, wherein the constitutively active variant is a gain of function mutation, a duplication, or a gene amplification. In any embodiment herein, it may be the cancer exhibits elevated expression and/or activity of at least one of PLA2G7A and PAFAH2 compared to healthy control cells.

“Effective amount” refers to the amount of a compound or composition required to produce a desired effect. In any embodiment and/or aspect disclosed herein (for simplicity's sake, hereinafter recited as “in any embodiment disclosed herein” or the like), the effective amount may be determined in relation to a subject. As used herein, a “subject” or “patient” is a mammal, such as a cat, dog, rodent or primate. Typically the subject is a human, and, preferably, a human suffering from or suspected of suffering from a cancer, a tumor, and/or a neoplasm. The term “subject” and “patient” can be used interchangeably. One example of an effective amount includes amounts or dosages that yield acceptable toxicity and bioavailability levels for therapeutic (pharmaceutical) use including, but not limited to, reduction of a tumor mass. In any embodiment disclosed herein, the effective amount may be an amount effective in treating a cancer, treating a tumor, shrinking a tumor, treating a neoplasm, shrinking a neoplasm, and/or increasing subject survival. By way of example, the effective amount of any embodiment herein including a compound of the present technology may be from about 0.01 μg to about 200 mg of the compound (such as about 160 mg of the compound). As another example, the effective amount of a compound of the present technology may be (in terms of mass of the compound/mass of patient) from 1×10⁻⁵ g/kg to 1 g/kg, 1×10⁻³ g/kg to 1.0 g/kg, 0.01 mg/kg to 100 mg/kg, 0.01 mg/kg to about 20 mg/kg, or, preferably, from 0.25 mg/kg to 10 mg/kg—thus, in any embodiment disclosed herein, the effective amount a compound of the present technology may be about 0.01 mg/kg, about 0.1 mg/kg, about 0.15 mg/kg, about 0.2 mg/kg, about 0.25 mg/kg, about 0.3 mg/kg, about 0.4 mg/kg, about 0.5 mg/kg, about 0.6 mg/kg/about 0.7 mg/kg, about 0.8 mg/kg, about 0.9 mg/kg, about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 65 mg/kg, about 70 mg/kg, about 75 mg/kg, about 80 mg/kg, about 85 mg/kg, about 90 mg/kg, about 95 mg/kg, about 100 mg/kg, or any range including and/or in between any two of these values (such as, e.g., about 0.25 mg/kg to about 10 mg/kg).

A method of any embodiment disclosed herein may comprise administering a pharmaceutical composition to the subject, where the pharmaceutical composition includes the effective amount of the compound of the present technology as well as a pharmaceutically acceptable carrier or one or more excipients, fillers or agents (collectively referred to hereafter as “pharmaceutically acceptable carrier” unless otherwise indicated and/or specified). Thus, the present technology also provides pharmaceutical compositions and medicaments including a compound of any embodiment disclosed herein and a pharmaceutically acceptable carrier. The compositions may be used in the methods and treatments described herein (for ease of reference, the medicaments and pharmaceutical compositions of the present technology may collectively be referred to herein as “compositions” or “compositions of the present technology” or the like). The pharmaceutical composition may be packaged in unit dosage form. The unit dosage form is effective in treating a tumor by reducing a tumor when administered to a subject in need thereof. Generally, a unit dosage including a compound of the present technology will vary depending on patient considerations. Such considerations include, for example, age, protocol, condition, sex, extent of disease, contraindications, concomitant therapies and the like. An exemplary unit dosage based on these considerations may also be adjusted or modified by a physician skilled in the art. For example, a unit dosage for a patient comprising a compound of the present technology may vary from 1×10⁻⁴ g/kg to 1 g/kg, preferably, 1×10⁻³ g/kg to 1.0 g/kg. Dosage of a compound of the present technology may also vary from (in terms of mass of the compound/mass of patient) 1×10⁻⁵ g/kg to 1 g/kg, 1×10⁻³ g/kg to 1.0 g/kg, 0.01 mg/kg to 100 mg/kg, 0.01 mg/kg to about 20 mg/kg, or, preferably, from 0.25 mg/kg to 10 mg/kg—thus, in any embodiment disclosed herein, the dosage may be about 0.01 mg/kg, about 0.1 mg/kg, about 0.15 mg/kg, about 0.2 mg/kg, about 0.25 mg/kg, about 0.3 mg/kg, about 0.4 mg/kg, about 0.5 mg/kg, about 0.6 mg/kg/about 0.7 mg/kg, about 0.8 mg/kg, about 0.9 mg/kg, about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 65 mg/kg, about 70 mg/kg, about 75 mg/kg, about 80 mg/kg, about 85 mg/kg, about 90 mg/kg, about 95 mg/kg, about 100 mg/kg, or any range including and/or in between any two of these values (such as, e.g., about 0.25 mg/kg to about 10 mg/kg). Suitable unit dosage forms, include, but are not limited to parenteral solutions, oral solutions, powders, tablets, pills, gelcaps, capsules, lozenges, suppositories, patches, nasal sprays, injectables, implantable sustained-release formulations, mucoadherent films, topical varnishes, lipid complexes, liquids, etc.

The pharmaceutical compositions and medicaments may be prepared by mixing one or more compounds of the present technology with pharmaceutically acceptable carriers, excipients, binders, diluents or the like. Such compositions can be in the form of, for example, granules, powders, tablets, capsules, syrup, suppositories, injections, emulsions, elixirs, suspensions or solutions. The instant compositions can be formulated for various routes of administration, for example, by oral, parenteral, topical, rectal, nasal, vaginal administration, or via implanted reservoir. Parenteral or systemic administration includes, but is not limited to, subcutaneous, intravenous, intraperitoneal, and intramuscular, injections. The following dosage forms are given by way of example and should not be construed as limiting the instant present technology.

For oral, buccal, and sublingual administration, powders, suspensions, granules, tablets, pills, capsules, gelcaps, and caplets are acceptable as solid dosage forms. These can be prepared, for example, by mixing one or more compounds of the instant present technology, or pharmaceutically acceptable salts or tautomers thereof, with at least one additive such as a starch or other additive. Suitable additives are sucrose, lactose, cellulose sugar, mannitol, maltitol, dextran, starch, agar, alginates, chitins, chitosans, pectins, tragacanth gum, gum arabic, gelatins, collagens, casein, albumin, synthetic or semi-synthetic polymers or glycerides. Optionally, oral dosage forms can contain other ingredients to aid in administration, such as an inactive diluent, or lubricants such as magnesium stearate, or preservatives such as paraben or sorbic acid, or anti-oxidants such as ascorbic acid, tocopherol or cysteine, a disintegrating agent, binders, thickeners, buffers, sweeteners, flavoring agents or perfuming agents. Tablets and pills may be further treated with suitable coating materials known in the art.

Liquid dosage forms for oral administration may be in the form of pharmaceutically acceptable emulsions, syrups, elixirs, suspensions, and solutions, which may contain an inactive diluent, such as water. Pharmaceutical formulations and medicaments may be prepared as liquid suspensions or solutions using a sterile liquid, such as, but not limited to, an oil, water, an alcohol, and combinations of these. Pharmaceutically suitable surfactants, suspending agents, emulsifying agents, may be added for oral or parenteral administration, and may include natural and modified cyclodextrin compounds.

As noted above, suspensions may include oils. Such oils include, but are not limited to, peanut oil, sesame oil, cottonseed oil, corn oil and olive oil. Suspension preparation may also contain esters of fatty acids such as ethyl oleate, isopropyl myristate, fatty acid glycerides and acetylated fatty acid glycerides. Suspension formulations may include alcohols, such as, but not limited to, ethanol, isopropyl alcohol, hexadecyl alcohol, glycerol and propylene glycol. Ethers, such as but not limited to, poly(ethyleneglycol), petroleum hydrocarbons such as mineral oil and petrolatum; aprotic solvents such as dimethyl sulfoxide; and/or water may also be used in suspension formulations.

Injectable dosage forms generally include aqueous suspensions or oil suspensions which may be prepared using a suitable dispersant or wetting agent and a suspending agent. Injectable forms may be in solution phase or in the form of a suspension, which is prepared with a solvent or diluent. Acceptable solvents or vehicles include sterilized water, Ringer's solution, or an isotonic aqueous saline solution. Alternatively, sterile oils may be employed as solvents or suspending agents. Typically, the oil or fatty acid is non-volatile, including natural or synthetic oils, fatty acids, mono-, di- or tri-glycerides.

For injection, the pharmaceutical formulation and/or medicament may be a powder suitable for reconstitution with an appropriate solution as described above. Examples of these include, but are not limited to, freeze dried, rotary dried or spray dried powders, amorphous powders, granules, precipitates, or particulates. For injection, the formulations may optionally contain stabilizers, pH modifiers, surfactants, bioavailability modifiers and combinations of these.

Compounds of the present technology may be administered to the lungs by inhalation through the nose or mouth. Suitable pharmaceutical formulations for inhalation include solutions, sprays, dry powders, or aerosols containing any appropriate solvents and optionally other compounds such as, but not limited to, stabilizers, antimicrobial agents, antioxidants, pH modifiers, surfactants, bioavailability modifiers and combinations of these. The carriers and stabilizers vary with the requirements of the particular compound, but typically include nonionic surfactants (Tweens, Pluronics, or polyethylene glycol), innocuous proteins like serum albumin, sorbitan esters, oleic acid, lecithin, amino acids such as glycine, buffers, salts, sugars or sugar alcohols. Aqueous and nonaqueous (e.g., in a fluorocarbon propellant) aerosols are typically used for delivery of compounds of the present technology by inhalation.

Dosage forms for the topical (including buccal and sublingual) or transdermal administration of compounds of the present technology include powders, sprays, ointments, pastes, creams, lotions, gels, solutions, and patches. The active component may be mixed under sterile conditions with a pharmaceutically-acceptable carrier or excipient, and with any preservatives, or buffers, which may be required. Powders and sprays can be prepared, for example, with excipients such as lactose, talc, silicic acid, aluminum hydroxide, calcium silicates and polyamide powder, or mixtures of these substances. The ointments, pastes, creams and gels may also contain excipients such as animal and vegetable fats, oils, waxes, paraffins, starch, tragacanth, cellulose derivatives, polyethylene glycols, silicones, bentonites, silicic acid, talc and zinc oxide, or mixtures thereof. Absorption enhancers can also be used to increase the flux of the compounds of the present technology across the skin. The rate of such flux can be controlled by either providing a rate controlling membrane (e.g., as part of a transdermal patch) or dispersing the compound in a polymer matrix or gel.

Besides those representative dosage forms described above, pharmaceutically acceptable excipients and carriers are generally known to those skilled in the art and are thus included in the instant present technology. Such excipients and carriers are described, for example, in “Remingtons Pharmaceutical Sciences” Mack Pub. Co., New Jersey (1991), and “Remington: The Science and Practice of Pharmacy,” 20^{th} Edition, Editor: Alfonso R Gennaro, Lippincott, Williams & Wilkins, Baltimore (2000), each of which is incorporated herein by reference.

The formulations of the present technology may be designed to be short-acting, fast-releasing, long-acting, and sustained-releasing as described below. Thus, the pharmaceutical formulations may also be formulated for controlled release or for slow release.

The instant compositions may also comprise, for example, micelles or liposomes, or some other encapsulated form, or may be administered in an extended release form to provide a prolonged storage and/or delivery effect. Therefore, the pharmaceutical formulations and medicaments may be compressed into pellets or cylinders and implanted intramuscularly or subcutaneously as depot injections or as implants such as stents. Such implants may employ known inert materials such as silicones and biodegradable polymers.

In any embodiment herein, specific dosages may be adjusted depending on conditions of disease, the age, body weight, general health conditions, sex, and diet of the subject, dose intervals, administration routes, excretion rate, and combinations of drugs. Any of the above dosage forms containing effective amounts are well within the bounds of routine experimentation and therefore, well within the scope of the instant present technology. Those skilled in the art are readily able to determine an effective amount by simply administering a compound of the present technology to a patient in increasing amounts until, for example, there is a reduction in the mass of a tumor in a subject. The compounds of the present technology can be administered to a patient at dosage levels in the range of about 0.1 to about 1,000 mg per day (discussed in further detail supra). For a normal human adult having a body weight of about 70 kg, a dosage in the range of about 0.01 to about 100 mg per kg of body weight per day is sufficient (discussed in further detail supra). The specific dosage used, however, can vary or may be adjusted as considered appropriate by those of ordinary skill in the art. For example, the dosage can depend on a number of factors including the requirements of the patient, the severity of the cancer associated with the tumor, and the pharmacological activity of the compound being used. The determination of optimum dosages for a particular patient is well known to those skilled in the art. Various assays and model systems can be readily employed to determine the therapeutic effectiveness of the treatment according to the present technology. Effectiveness of the compositions (as well as determination of effective amounts) and methods of the present technology may also be demonstrated by a decrease in the mass of a tumor, slowing the growth of a tumor, and/or increasing subject survival. For each of the indicated conditions described herein, test subjects will exhibit a 10%, 20%, 30%, 50% or greater reduction, up to a 75-90%, or 95% or greater, reduction, in one or more symptom(s) caused by, or associated with, the disorder in the subject, compared to placebo-treated or other suitable control subjects.

In one aspect, a compound of the present technology is administered to a patient in an amount or dosage suitable for therapeutic use (e.g., included in a pharmaceutical composition of any embodiment of the present technology). Generally, a unit dosage comprising a compound of the present technology will vary depending on patient considerations. Such considerations include, for example, age, protocol, condition, sex, extent of disease, contraindications, concomitant therapies and the like. An exemplary unit dosage based on these considerations can also be adjusted or modified by a physician skilled in the art. For example, a unit dosage for a patient comprising a compound of the present technology can vary from 1×10⁻⁴ g/kg to 1 g/kg, preferably, 1×10⁻³ g/kg to 1.0 g/kg. Dosage of a compound of the present technology can also vary from 0.01 mg/kg to 100 mg/kg or, preferably, from 0.1 mg/kg to 10 mg/kg.

A compound of the present technology can also be modified, for example, by the covalent attachment of an organic moiety or conjugate to improve pharmacokinetic properties, toxicity or bioavailability (e.g., increased in vivo half-life). The conjugate can be a linear or branched hydrophilic polymeric group, fatty acid group or fatty acid ester group. A polymeric group can comprise a molecular weight that can be adjusted by one of ordinary skill in the art to improve, for example, pharmacokinetic properties, toxicity or bioavailability. Exemplary conjugates can include a polyalkane glycol (e.g., polyethylene glycol (PEG), polypropylene glycol (PPG)), carbohydrate polymer, amino acid polymer or polyvinyl pyrolidone and a fatty acid or fatty acid ester group, each of which can independently comprise from about eight to about seventy carbon atoms. Conjugates for use with a compound of the present technology can also serve as linkers to, for example, any suitable substituents or groups, radiolabels (marker or tags), halogens, proteins, enzymes, polypeptides, other therapeutic agents (for example, a pharmaceutical or drug), nucleosides, dyes, oligonucleotides, lipids, phospholipids and/or liposomes. In one aspect, conjugates can include polyethylene amine (PEI), polyglycine, hybrids of PEI and polyglycine, polyethylene glycol (PEG) or methoxypolyethylene glycol (mPEG). A conjugate can also link a compound of the present technology to, for example, a label (fluorescent or luminescent) or marker (radionuclide, radioisotope and/or isotope) to comprise a probe of the present technology. Conjugates for use with a compound of the present technology can, in one aspect, improve in vivo half-life. Other exemplary conjugates for use with a compound of the present technology as well as applications thereof and related techniques include those generally described by U.S. Pat. No. 5,672,662, which is hereby incorporated by reference herein.

In any embodiment disclosed herein, the cancer may be (and/or the tumor may be of a cancer such as, and/or the neoplasm may be of a cancer such as) squamous cell carcinoma, soft tissue sarcoma, oral melanoma, acute lymphoblastic leukemia (ALL), acute myeloid leukemia (AML), adrenocortical carcinoma, AIDS-related cancers, Kaposi sarcoma (soft tissue sarcoma), AIDS-related lymphoma (lymphoma), anal cancer, appendix cancer, gastrointestinal carcinoid tumors, astrocytomas, atypical teratoid/rhabdoid tumor, basal cell carcinoma of the skin, bile duct cancer, bladder cancer, bone cancer (includes Ewing Sarcoma and Osteosarcoma and Malignant Fibrous Histiocytoma), brain tumors, breast cancer, bronchial tumors (lung cancer), Burkitt lymphoma, carcinoid tumor (gastrointestinal), carcinoma of unknown primary, cardiac (heart) tumors, childhood brain cancer, germ cell tumor, primary CNS lymphoma, cervical cancer, cholangiocarcinoma, chordoma, chronic lymphocytic leukemia (CLL), chronic myelogenous leukemia (CIVIL), chronic myeloproliferative neoplasms, colorectal cancer, craniopharyngioma, cutaneous T-cell lymphoma, ductal carcinoma in situ (DCIS), embryonal tumors, medulloblastoma, endometrial cancer (uterine cancer), ependymoma, esophageal cancer, esthesioneuroblastoma (head and neck cancer), extracranial germ cell tumor, eye cancer, retinoblastoma, fallopian tube cancer, fibrous histiocytoma of bone, osteosarcoma, gallbladder cancer, gastric (stomach) cancer, gastrointestinal stromal tumors (GIST) (soft tissue sarcoma), germ cell tumors, childhood central nervous system germ cell tumors (brain cancer), childhood extracranial germ cell tumors, extragonadal germ cell tumors, ovarian germ cell tumors, testicular cancer, gestational trophoblastic disease, hairy cell leukemia, head and neck cancer, heart tumors, hepatocellular (liver) cancer, histiocytosis, Hodgkin lymphoma, intraocular melanoma, islet cell tumors, pancreatic neuroendocrine tumors, kidney (renal cell) cancer, Langerhans cell histiocytosis, laryngeal cancer (head and neck cancer), leukemia, lip and oral cavity cancer (head and neck cancer), liver cancer, lung cancer, lymphoma, male breast cancer, malignant fibrous histiocytoma of bone and osteosarcoma, melanoma, Merkel cell carcinoma (skin cancer), mesothelioma, metastatic cancer, metastatic squamous neck cancer with occult primary (head and neck cancer), midline tract carcinoma with nut gene changes, mouth cancer (head and neck cancer), multiple endocrine neoplasia syndromes, multiple myeloma/plasma cell neoplasms, mycosis fungoides (lymphoma), myelodysplastic syndromes, myelogenous leukemia, myeloid leukemia, myeloproliferative neoplasms, nasal cavity and paranasal sinus cancer (head and neck cancer), nasopharyngeal cancer (head and neck cancer), neuroblastoma, non-Hodgkin lymphoma, non-small cell lung cancer, oral cancer, osteosarcoma and malignant fibrous histiocytoma of bone, ovarian cancer, pancreatic cancer, pancreatic neuroendocrine tumors (islet cell tumors), papillomatosis (childhood laryngeal), paraganglioma, paranasal sinus and nasal cavity cancer (head and neck cancer), parathyroid cancer, penile cancer, pharyngeal cancer (head and neck cancer), pheochromocytoma, pituitary tumor, plasma cell neoplasm/multiple myeloma, pleuropulmonary blastoma (lung cancer), pregnancy and breast cancer, primary central nervous system (CNS) lymphoma, primary peritoneal cancer, prostate cancer, rectal cancer, recurrent cancer, renal cell (kidney) cancer, rhabdomyosarcoma, salivary gland cancer (head and neck cancer), sarcoma, childhood rhabdomyosarcoma, childhood vascular tumors, Ewing sarcoma (bone cancer), Kaposi sarcoma, osteosarcoma (bone cancer), Sézary syndrome (lymphoma), skin cancer, small cell lung cancer, small intestine cancer, squamous cell carcinoma of the skin, squamous neck cancer with occult primary, metastatic (head and neck cancer), stomach (gastric) cancer, T-cell lymphoma, throat cancer (head and neck cancer), oropharyngeal cancer, hypopharyngeal cancer, thymoma and thymic carcinoma, thyroid cancer, tracheobronchial tumors (lung cancer), transitional cell cancer of the renal pelvis and ureter (kidney (renal cell) cancer), urethral cancer, uterine cancer, uterine sarcoma, vaginal cancer, vascular tumors, vulvar cancer, and/or Wilms tumor and other childhood kidney tumors. In any embodiment disclosed herein, the cancer may be (and the tumor may be of a cancer such as, and the neoplasm may be of a cancer such as) a pancreatic cancer, a colorectal cancer, a hepatocellular cancer, a bile duct cancer, a soft tissue sarcoma, a blood or hematopoietic cell cancer, a breast cancer, a lung cancer, a uterine or cervical cancer, a thyroid cancer, a bladder cancer, a kidney cancer, a gastric cancer, an ovarian cancer, a brain cancer, a mesothelioma cancer, a skin cancer, a head and neck cancer, a neuroendocrine cancer or neoplasm, an esophagus cancer, a testicular cancer, a prostate cancer, or a thymus cancer. In any embodiment disclosed herein, the cancer may include (and/or the tumor may be of a cancer including, and/or the neoplasm may be of a cancer including) an adenocarcinoma, a uterine carcinoma, a squamous cell carcinoma, small cell carcinoma, a transitional carcinoma, a serous carcinoma, a clear-cell carcinoma, a mucinous adenocarcinoma, an undifferentiated carcinoma, a dedifferentiated carcinoma, a serous adenocarcinoma, or a combination of any two or more thereof.

In any embodiment herein, the administering may include local administration of the compound to a site in the subject including the cancer (such as a tumor) or local administration of the composition to a site in the subject including the cancer (such as a tumor). In any embodiment herein, the administering may include oral, rectal, nasal, vaginal, transdermal, intravenous, intramuscular, or inhalation administration. In any embodiment herein, the administering may include injection of the compound into the site in the subject including the cancer (such as a tumor) or proximal to the site in the subject including the cancer (such as a tumor).

The compounds of the present technology may also be administered to a patient along with other conventional therapeutic agents that may be useful in the treatment of tumors or in vaccination. The administration may include oral administration, parenteral administration, or nasal administration. In any of these embodiments, the administration may include subcutaneous injections, intravenous injections, intraperitoneal injections, or intramuscular injections. In any of these embodiments, the administration may include oral administration. The methods of the present technology can also include administering, either sequentially or in combination with one or more compounds of the present technology, a conventional therapeutic agent in an amount that can potentially or synergistically be effective for the treatment of tumors or in vaccination. In any embodiment herein, the administering may further include administration of a chemotherapeutic agent such as an alkylating agent; a nitrosourea; an antimetabolite; an anthracycline; a topoisomerase II inhibitor; a mitotic inhibitor; an anti-estrogen; a progestin; an aromatase inhibitor; an anti-androgen; an LHRH agonist; a corticosteroid hormone; a DNA alkylating agent; a taxane; a vinca alkaloid; a microtubule poison, or a combination of any two or more thereof. In any embodiment herein, the administering may further include administration of a chemotherapeutic agent such as busulfan, cisplatin, carboplatin, oxaliplatin, an octahedral platinum (IV) compound, chlorambucil, cyclophosphamide, ifosfamide, dacarbazine (DTIC), mechlorethamine (nitrogen mustard), melphalan, temozolomide, carmustine (BCNU), lomustine (CCNU), 5-fluorouracil, capecitabine, 6-mercaptopurine, methotrexate, gemcitabine, cytarabine (ara-C), fludarabine, pemetrexed, daunorubicin, doxorubicin (Adriamycin), epirubicin, idarubicin, mitoxantrone, topotecan, irinotecan, etoposide (VP-16), teniposide, paclitaxel, docetaxel, vinblastine, vincristine, vinorelbine, prednisone, dexamethasone, L-asparaginase, dactinomycin, thalidomide, tretinoin, imatinib (Gleevec), gefitinib (Iressa), erlotinib (Tarceva), rituximab (Rituxan), bevacizumab (Avastin), ipilimumab, nivolumab (Opdivo), pembrolizumab (Ketruda), tamoxifen, fulvestrant, anastrozole, exemestane, letrozole, megestrol acetate, bicalutamide, flutamide, leuprolide, goserelin, or a combination of any two or more thereof. In any embodiment herein, the administering of the chemotherapeutic agent may include local administration of the chemotherapeutic agent to a site in the subject including the cancer. In any embodiment herein, the administering of the chemotherapeutic agent may include oral, rectal, nasal, vaginal, transdermal, intravenous, intramuscular, or inhalation administration. In any embodiment herein, the administering of the chemotherapeutic agent may include injection of the chemotherapeutic agent into the site in the subject including the cancer or proximal to the site in the subject including the cancer.

The examples herein are provided to illustrate advantages of the present technology and to further assist a person of ordinary skill in the art with preparing or using the compounds and compositions of the present technology. The examples herein are also presented in order to more fully illustrate the preferred aspects of the present technology. The examples should in no way be construed as limiting the scope of the present technology, as defined by the appended claims. The examples can include or incorporate any of the variations, aspects, or embodiments of the present technology described above. The variations, aspects, or embodiments described above may also further each include or incorporate the variations of any or all other variations, aspects, or embodiments of the present technology.

### EXAMPLES

Statistical Analysis and Software. All p values were calculated using two-tailed unpaired t-tests with a 95% confidence interval using GraphPad Prism 7.03. Multiple comparisons were made using one-way ANOVA corrected by Holm-Sidak. N-replicates and error are reported in each figure. Throughout, ns=not significant, *p<0.05, **p<0.01, ***p<0.001, ****p<0.0001. Proliferation curves were generated using GraphPad Prism 7.03; all other data including nano-reporter distribution fits to Gaussian functions were analyzed using OriginPro 9.0. MATLAB R2014a was used to generate color pixel maps, image overlays, and raw reporter data corrections. ImageJ or Fiji (NIH) was used to analyze western blot film densities and to generate blot and immunofluorescence images, β-gal images, and wide field/confocal images (HDF5/Bio-Formats plugins). Chemical structures and cartoons were designed with ChemBioDraw Ultra 13.0. Figure and scheme layouts were designed with Adobe Illustrator CS6 v16.0.3.

Cell Lines. Primary mouse embryo fibroblasts (MEF) were derived from Lowe laboratory C57BL/6 mouse colonies at MSKCC. MEF were immortalized by SV40 large T antigen.^{82, 83} Immortalized MEF were transduced by retrovirus packaged by a HEK293T line transfected with cDNA plasmids. cDNA inserts, empty (scramble) vector, mouse K-ras^{G12V} 4B, or human H-ras^{G12V} were cloned in front of a Tet-ON 3G pTURN construct⁸⁴ using a modified version of the retroviral plasmid vector pTRE-Tight (Clonetech).⁸⁵ Plasmids were co-transfected with retrovirus packaging plasmids in HEK293T cells using Lipofectamine 2000 (Life Technologies) and chloroquine/BSA supplementation to enhance viral load; fresh media was added after 16 h, and viral supernatants were collected 2 days after transfection. Retroviral transduction occurred through exposure of 0.45 μm filtered HEK conditioned medium (plus 4 μg/mL polybrene, Fisher TR1003G) to 6E5 target cells. Hygromycin (150 μg/mL, ThermoFisher 10687-010) was used for selection. A K-ras^{G12D} knock-in line was prepared with self-excising retroviral Cre-recombinase^{19, 84, 86} prior to infection with SV40LT. SV40LT MEF harboring constitutive Akt1 or constitutive short-guide RNA against Pten were described previously.⁸⁶ The SV40LT Atg5 knock-out line⁸² was transduced by the pTURN plasmid. All cell lines were tested for mycoplasma prior to expansion and storage. RAW 264.7 mouse macrophage/monocyte were from ATCC (TIB-71) and cultured in medium as indicated in the main text, but with the addition of 10% heat inactivated FBS. Malignant lines were cultured using the same medium and were gifts from research groups at Memorial Sloan Kettering Cancer Center and included the following: KP Lung, KrasG12D/p53R270H invasive primary lung adenocarcinoma from mouse; Human PA-TU-8988T pancreatic adenocarcinoma, Human HCT-116 colon carcinoma, Human A549 lung adenocarcinoma, Human Panc-1 pancreatic ductal epithelioid adenocarcinoma.

Cell Culture. Cell cultures were incubated at 37° C. with 5% CO₂ and ambient oxygen. Complete culture medium used in this work consisted of pyruvate supplemented DMEM (ThermoFisher 10313021) and the following final concentrations of additives: 4.75% v/v heat inactivated fetal bovine serum (FBS) (ThermoFisher 10082147), 88.5 U/mL Pen Strep (ThermoFisher 15140122), 3.54 mM GlutaMAX (ThermoFisher 35050061), 1.77 mM L-glutamine (ThermoFisher 25030081), and 35 mM HEPES (ThermoFisher 15630080). Routine passage was performed with TrypLE (ThermoFisher 12604013). Cell lines were not used after seven to eight passages out from liquid nitrogen storage; experiments were started after at least two passages out from liquid nitrogen storage. Cultured cells were routinely split prior to 80-90% confluence. To supplement lipophilic drugs, the DMSO dissolved stock compound was pre-incubated with the appropriate volume of 37° C. complete culture medium and gently mixed for 10 minutes. For lipoprotein deficient experiments, FBS was replaced with the same final % LPDS (Sigma S5394). All pre-made solutions were sterile filtered (0.22 μm). Unless indicated otherwise, doxycycline (500 ng/mL) was added into media simultaneously with other test compounds. To supplement fatty acids, a volume of fatty acid oil needed to make a 6 mM stock in 1 mL was transferred to a sterile Eppendorf purged with nitrogen gas. To this tube, 1 mL of 1 mM delipidated BSA (Fraction V fatty acid free BSA, Sigma A7030, Lot #5LBQ0873V) was added. The tube was placed into an auto-mixer at 1000 rpm, 37° C., for 30 minutes (Eppendorf ThermoMixer C). The resulting stock PUFA-BSA was sterile filtered with a 0.22 μm PES membrane. The stock was aliquoted, nitrogen purged, and stored at −20° C. until use. Culture medium supplemented with the PUFA mix contained 5 μM docosahexanoic acid (Sigma D2534), 5 μM arachidonic acid (EMD 181198), and 2.5 μM each of linoleic acid (LA) (Sigma L1376) and linolenic acid (ALA) (Sigma L2376). Delipidated BSA was supplemented in the control condition.

Proliferation Assays. To assay proliferation, 6E4 cells/well were plated in 6-well plates (ThermoFisher 140675) with complete medium to generate triplicate wells, per experimental condition, for Day 0 through Day 3/4. 6-8 h after seeding cells, wells were briefly rinsed with fresh medium and then cultured with the appropriately supplemented test medium. The Day 0 plate was counted at this time. Plates were counted at 24-h intervals. Attached and spread MEF's are >95% viable,⁸⁴ as confirmed by a lack of trypan blue staining or positive western blot for cleaved caspase 7 or PARP (data not shown). To count attached cells, media was aspirated from each well then rinsed, using manual agitation, twice in room temperature (RT) 1×HBSS (w/o Ca/Mg++). Attached cells were trypsinized with 1 mL TrypLE. An imaging cytometer (Thermo T10796; factory default settings) was loaded with chamber slides (ThermoFisher T10794) containing 24 μL of the trypsinized cell suspension. 18 fields were imaged; the cytometer output was given in cells/mL. Output was gated for cell/particle size so that the final count did not include particles at or below 7 μm, which was taken to reflect background debris from culture. An upper gating threshold was set at 34 μm to include size-doubling of the measured 17 μm average.

Hyperspectral Microscopy. For lipid reporter imaging experiments, cells were plated on 35 mm glass bottom dishes (MatTek Corp. P35G-1.5-10-C) so that around 0.5-0.8E6 cells were present on assay day. 24 h prior to assay, cells were briefly rinsed with fresh media and cultured as described. On the day of the assay, test media was removed to a sterile Eppendorf, spun in a mini-fuge to pellet cellular debris, and stored at 37° C. Attached cells were briefly rinsed with Pulse media (serum-free complete medium), then exposed to nano-reporter (0.2 μg/mL in Pulse media) for 30 minutes. After exposure, excess nano-reporter was removed by rinsing in complete medium and the stored test media was re-added to the dish for the 5-h incubation period. Near-infrared hyperspectral microscopy⁸⁷ was performed on live attached cells to obtain fluorescence emission maps from endomembrane vesicles.⁸⁸ Briefly, a 730 nm continuous wave diode laser was pumped through fibers to a 100× oil objective to excite the fluorescent reporters inside attached cells. Collected emission was stored as a hyperspectral imaging cube of encoded wavelengths rectified between 1100 and 1200 nm. The center wavelength for each pixel in the cube was obtained by fitting a Lorentzian function in MATLAB. The center wavelength of the entire population of pixels in each cube, representing a 100× magnified imaging field, was fit with a Gaussian function in OriginPro 9.0. The resulting fit center wavelength values are reported. In the main Figure histogram, (*) represents emission values from immobile sensors located at the cell periphery that were not included in fitting analysis.

Well Plate Spectroscopy. Ensemble spectroscopic measurements were performed with a custom-built near-infrared spectrometer.⁸⁹ Lipid stocks were pre-dissolved in methanol and equilibrated in aqueous buffer for 3 h prior to sensor addition (1 μg/mL sensor in 150 buffer). The sensor-lipid mixtures were incubated for 5 h at 37° C. then moved to a 96-well plate. Each row contained a blanking well. The sample wells were excited by a supercontinuum laser in 3 nm steps from 500 nm to 839 nm using a 1 second exposure time. Emission from 930 nm to 1368 nm was acquired and data was corrected and fit to a Lorentzian function in MATLAB to generate peak intensity and center wavelength.

Immunocytochemistry. Cells were plated onto chamber slides (Millipore PEZGS0416/PEZGS0816) and treated as described. At the time of assay, cells were fixed for 15 minutes at RT by 3.7% paraformaldehyde (using a 4° C. 37% stock added dropwise to the warm culture). Fixative was inactivated by 10-minute incubation with 75 mM ammonium chloride in 1×PBS. After two rinses with 1×PBS, chambers were stored at 4° C. or processed immediately (all reagent buffers were prepared using a 1×PHEM buffer: 60 mM PIPES, 25 mM HEPES, 10 mM EGTA, 2 mM MgCl2, 5 mM NaCl, 70 mM KCl, pH 6.9): permeabilization with 0.1% Tween-20 (TW20) for 5 minutes RT, blocking with 5% BSA/0.3% TW20 for 3 h RT, then 4° C. overnight incubation with primary antibody in 5% BSA/0.3% TW20 with gentle rocking. After three RT washes for 5 minutes each, cells were blocked as above for 1-h RT after the addition of 5% Goat serum (ThermoFisher 016201). Fluorescent secondary antibody was diluted into 5% BSA/0.3% TW20/5% Goat serum and chambers were incubated for 1-h RT in the dark. In some cases, after three 10-minute RT washes, a diluted nuclear counterstain (Hoechst in PBS, ThermoFisher 62249) was applied for 2 minutes followed by a 2 minute wash. Mounting media (Life Technologies P36961) and coverslip (Fisher 1254418) were applied on each slide. The ICC protocol was modified depending on target: Nuclear—secondary incubation was increased to 2 h; non-nuclear endomembrane—reduction to 2% fixative, permeabilization for 10 minutes in 100% −20° C. methanol, followed by one rinse and blocking as described. The following primary antibodies were used: anti-HP1γ (1:100, Cell Signaling 2619S), anti-phospho-H2A.X (1:300, Cell Signaling 9718), anti-PLA2g7 (1:50, Proteintech 15526-1-AP), anti-PAFAH2 (1:50, Proteintech 10085-1-AP); secondary antibodies were chosen from the following: Alexa488/568 goat anti-mouse, Alexa568 anti-rabbit IgG (Life Technologies A11001/A11004 & A11011), goat anti-rabbit IgG Super Clonal 555/647 (Invitrogen A27040/A27039), or goat anti-rat IgG 555/647 (Life Technologies 21247/21434).

Immunoblotting. Protease and phosphatase inhibitor cocktail (Thermo 78446) supplemented with 100 μM pepstatin A (Sigma 11359053001) was diluted 1:100 into RIPA Buffer (Pierce 89901) or denaturing immunoprecipitation (IP) buffer without ethylmaleimide (20 mM HEPES, 50 mM NaCl, 0.5% NP-40, 1 mM EDTA, 0.5% SDS, 0.5% SDC) to make working cell lysis buffer for soluble targets (RIPA) or insoluble/membrane associated targets (IP buffer). Cells were washed twice with cold 1×HBSS. Ice cold lysis buffer was added, and cells were scrapped with a rubber policeman. Lysate was pooled into an Eppendorf on wet ice and set to mix for 30 minutes at 4° C. Lysate was homogenized through a 26G needle with 5 full strokes, and then spun at 16,000 rcf for 20 minutes in a 4° C. centrifuge. RT Bradford reagent (BioRad 5000205) was mixed 1:1 with deionized water diluted samples containing either a BSA standard the sample lysate. Absorbance was measured at 595 nm in a Tecan Infinite M1000 Pro plate reader. Loading buffer consisted of 1× Laemmli Buffer/2-Mercaptoethanol (BioRad 1610747/1610710) and deionized water if necessary. Final loading samples were aliquoted and stored at −80° C. until use. Frozen samples were immediately heated for 8 minutes at 75° C. (phospho-protein targets) or for 6 minutes at 90° C. (all other targets). TGX precast gels (BioRad 4568094, 4-20%) were loaded into a Tetra Cell electrophoresis apparatus (BioRad 1658004) filled with chilled 1× Tris/Glycine/SDS buffer (BioRad 1610732). 1.5 μL running ladder (LI-COR 92698000), 10-30 μL sample, or 10-30 μL blank sample buffer were injected into each lane. Electrophoresis was run at 110 Volts, CV, for 80 minutes or until appropriate ladder separation. Blot sandwiches comprised a 0.2 μm PVDF membrane (BioRad 1620174) pre-incubated in methanol followed by transfer buffer (BioRad 10026938) held between buffer wetted-stack paper (BioRad) and the gel. A Trans-Blot Turbo transfer system (BioRad 1704150) was used on the 7-minute Midi-Turbo setting. Blot membranes were cut and immediately rinsed in 1×TBS, then moved into a blocking solution (3% w/v BSA in 1×TBS-T) (BioRad 1706435; Sigma P1379) and agitated at RT for 1.5 h. Primary antibody was diluted into 1 mL of blocking buffer, which was sandwiched with the membrane between two parafilm sheets before sealing. Membrane sandwiches were incubated overnight at 4° C. with rotation. Membranes were peeled from the parafilm into 1×TBS-T and washed three times for 10 minutes at RT with agitation. HRP conjugated secondary antibody was diluted in enough blocking solution to cover the membrane for 1 h at RT with agitation. After three more 10-minute washes, membranes were rinsed in RT 1×TBS then incubated for 1 minute in HRP substrate (Millipore WBLUR0500). To expose film, moist membranes were placed inside plastic inserts in an x-ray cassette. Antibodies were as follows: anti-Cox1 (1:500, Cell Signaling 9896), anti-Cox2 (1:1000, Cell Signaling 12282), anti-p38 MAPK (1:1300, Cell Signaling 8690), anti-phospho-p38 MAPK T180/Y182 (1:500, Cell Signaling 4511), anti-ATF-2 (1:1000, Cell Signaling 9226), anti-phospho-ATF-2 T69/T71 (1:1000, Cell Signaling 5112), anti-TBP (1:1500, Cell Signaling 44059), anti-cPLA2 (1:1000, Cell Signaling 5249), anti-phospho-cPLA2 S505 (1:750, Cell Signaling 53044), anti-phospho-IκBα S32 (1:500, Cell Signaling 2859), anti-IκBα (1:1000, Cell Signaling 4812) anti-NF-κB p65 (1:1000, Selleckchem.com A5075), anti-phospho-NF-κB p65 S468 (1:1000, Cell Signaling 3039), anti-phospho-NF-κB p65 S536 (1:1000, Cell Signaling 3033), anti-TNF-α (1:1000, Cell Signaling 11948), anti-IL-6 (1:500, Cell Signaling 12912), anti-prostaglandin E synthase (1:1000, Abcam ab180589), anti-p44/42 MAPK (1:1000, Cell Signaling 4695), anti-phospho-p44/42 MAPK (1:1000, Cell Signaling 9101), anti-p16 (1 Abcam 189034), anti-p21^{waf1/cip1} (1.6 Abcam 109199), anti-SAPK/JNK (1:1000; Cell Signaling 9252), anti-phospho-SAPK/JNK T183/Y185 (1:500; Cell Signaling 4668), anti-p19ARF (1:1000, Abcam ab80), anti-PLA2g7 (1:400, Proteintech 15526-1-AP), anti-PAFAH2 (1:400, Proteintech 10085-1-AP); secondary antibody was an HRP-conjugated anti-rabbit (1:2000-1:5000, Cell Signaling 7074).

SA-β-Galactosidase Staining. Staining was performed per the manufacturer's instructions (Cell Signaling 9860) using cells seeded into 6 well plates. After processing, a concentrated Hoechst dye solution was added into each well to counterstain nuclei and cause cytosolic bleed through on high-gain fluorescence imaging. For analysis and reporting, the RGB images acquired by the color camera on the microscope were deconvolved into three H&E DAB components using the Color Deconvolution plugin in Fiji. Only Color 1, represented by (R=0.650, G=0.704, B: 0.286), was used for analysis. An identical threshold was applied to the resulting single-color images (8-bit) to obtain binary masks. These binary images were the β-Gal positive mask. The total cell mask was generated separately using a segmentation program on the DAPI images, acquired from the same field, after scaling was used to increase the intensity of cytosolic bleed-through. Manual inspection showed the segmentation was accurate. The fraction (and reported %) of β-Gal positive cells in each field was calculated as: #segmented regions overlaying with β-Gal/total #nuclei. The technical replicates (5-10 per condition) were combined for each biological replicate, which were used to determine the % positive mean and standard deviation. The representative images in each Figure are color deconvolved transmitted light images, using the Azan Mallory setting in Fiji, highlighting the β-Gal stain.

Pan-sPLA2/PAF-AH Activity and Total Lipid Hydroperoxides. For chromogenic assay, commercial enzyme activity-based kits for sPLA2 (Cayman 765001), PAF-AH assay (Cayman 760901), or Lipid hydroperoxides (LPO) (Cayman 705003) were used per manufacturer instructions. Required water and solvents were LC-MS grade. Briefly, cells were induced so that on the day of the assay wells of a 6-well plate were 90%-100% confluent. For the sPLA2 assays, lysates/medium was spin filtered twice against cold PBS using a cold 10 kDa cut-off membrane device (Sigma UFC501096).

Lipidomics and Analysis. 100 mm plates were seeded, grown, and induced so that on the day of harvest dishes were 90-100% confluent. Culture medium was Phenol red-free. To harvest conditioned media, the full volume was moved to a glass vial on dry ice/alcohol slurry and a fresh cold PBS volume equal to ¼ of the collected volume was used to rinse the cells; this volume was collected and pooled. To harvest lysate, a separate dish of cells was washed three times with cold HBSS; on the last wash, liquid was aspirated, and the dish was placed onto the dry ice/alcohol slurry to freeze for 30 seconds. 1 mL of a 2:0.8 parts methanol:water mix (chilled to −80° C. and stored on dry ice) was added to the plate. The dish was immediately moved to wet ice and scrapped with a rubber policeman. Lysate was pooled in the bottom of the dish, and then transferred to a glass vial on dry ice until −80° C. storage. The lysate extract vials were briefly flushed with nitrogen gas. Modified Bligh-Dyer extraction was performed in a dedicated metabolomics laboratory. LC-MS analysis used an Agilent 6490 Triple Quadrupole MS integrated with Agilent 1260 Infinity UHPLC (normal phase for glycerophospholipid/sphingolipid; reverse phase for sterol/glycerolipid). Quantitation used an MRM method under positive and negative electrospray modes. Raw data was reported as Mol %, or ng/mL of the lipid species normalized to the absolute total lipid abundance (ng/mL) detected by the mass spectrometer. Reported data is Mol % divided by the total adherent cell count from a parallel-treated plate, calculated as a Fold change versus control: (iKRas/Vector)-1.

Confocal Microscopy. High resolution confocal scanning was performed with a point-scanning LSM 880 instrument using an AiryScan Module (Carl Zeiss), a 63×1.4 NA oil objective, and the appropriate laser lines and filters. For fixed cell experiments samples were mounted with #1.5 cover glass which was in contact with the objective. Exposure time and detector gain were held constant within experiments.

Wide Field Microscopy. Transmitted light and immunofluorescence images were acquired with an Olympus IX51 inverted microscope and an Olympus DP73 (color) or XM10 (grey scale) camera. An X-Cite series 120Q proprietary mercury vapor short arc excitation lamp provided illumination. Appropriate Ex/Em filter cubes were available. Exposure time and detector gain were held constant within experiments.

siRNA Knockdown. Transient knockdown of MEF lines was achieved by plating target cells to reach 80-90% confluence on the day of assay. For extended experiments, cells were plated to reach 40-50% confluence on treatment day. For each siRNA and target well, two Eppendorf tubes were each filled with 150 μL of serum- and antibiotic-free DMEM. The transfection cocktail was made by transferring siRNA into one tube such that the final volume in the target well was 45 nM. Into the other tube, 9 μL of RNAiMAX (Thermofisher 13778) was added. The siRNA volume was added to the RNAiMAX volume and mixed by pipetting. The cocktail was incubated at RT for 10 minutes and then added, dropwise, to a target well containing 2 mL of fresh complete medium. Plates were swirled to mix and incubated at 37° C. for 48 h. Doxycycline and other compounds were added after the first 24 h of exposure to siRNA: a 1 mL aliquot of the wells' contents was removed, mixed with the compounds, and returned. siRNA was as follows: Universal negative controls (Sigma SIC001/002 WDAA), p1a2g7 #1 (Sigma NM_013737 SASI_Mm01_00162678), p1a2g7 #2 (Sigma NM_013737 SASI_Mm01_00162677), pafah2 #1 (Sigma NM_133880 SASI_Mm01_00180435), pafah2 #2 (Sigma NM_133880 SASI_Mm01_00180436).

Lactate Dehydrogenase (LDH) Assay. The lactate dehydrogenase activity in culture conditioned medium was assayed using a commercial kit (ThermoFisher C20300). Briefly, at the end point after cell treatment and incubation plates were manually agitated to dislodge any loose cells. The conditioned medium was transferred to a 15 mL tube. 9% v/v Triton X-100 (TX100) in deionized water was added to each tube and vortexed. Fresh complete medium was vortexed with 9% Triton X-100 to make the negative control. To make the positive control (i.e. 100% lysis), one well of adherent cells was exposed a volume of fresh negative control mix, scraped, and returned to the tube. After being vortexed and incubated at RT for 5 minutes, all tubes were centrifuged (7,000 rcf at RT). An appropriate volume of the resulting supernatant was used in the manufacturer's 384-well format assay. Results (% Death) were calculated at each time point; the % Death ratio (final/initial time) is reported.

Cell Viability after darapladib treatment. 5,000 cells/well of doxycycline induced iKRas or target mouse/human cells were seeded in 96-well plates 1 day prior to the start of treatment. On the day of the experiment, increasing concentrations of darapladib were added in triplicate to the conditioned media. After incubation for 48 h, the cell viability was assessed using the CellTiter-Glo® luminescent assay (Promega G9681). Prior to use, reconstituted kit reagent was equilibrated to room temperature. To initiate cell lysis, 50 μL of reagent was added to each well. Plates were incubated for 10 minutes at room temperature with gentle mixing on an orbital shaker. Luminescence was measured using a Tecan Infinite M1000 Pro plate reader (Tecan Group Ltd.). Results from the assays were calculated as a percentage of the non-treated control. The reported EC₅₀ of darapladib ranges from 10-1000 nM, depending on the cell type and downstream marker used. The enzyme IC₅₀ ranges from 1-10 nM.

Cholera Toxin Subunit B (CTxB) Labeling. Cells were plated onto chamber slides or glass bottom dishes. Cells were pulsed as previously described with an Alexa647-ssDNA coated reporter alone or in combination with cholera toxin subunit B (CTxB, 0.5 μg) (ThermoFisher C22843). Media was removed and cells were triple rinsed in room temperature (RT) 1×HBSS. At the final time point, cells were fixed in situ by adding, dropwise, microscopy-grade paraformaldehyde (EMS 15714-S) onto culture medium and gently swirled to generate a final concentration of 2% v/v. In some cases, nuclear Hoechst counterstain was added into the fixative at a 1:10 dilution from stock. Imaging proceeded as indicated below. Confocal movies were acquired (data not shown) which showed correlated movement of vesicles labelled with CTxB and Alexa647 in the lumen.

Trans-Golgi Network 38-GFP Fusion Expression. For each 35 mm dish, 2 μg of cDNA, Tgolnl (Sino Biological MG5A1193-ACG) was added to serum- and antibiotic-free DMEM containing 2 μL PLUS reagent. 8 μL Lipofectamine LTX (Thermofisher 15338030) was added to another tube of serum-free medium. Tubes were mixed by pipetting and left to incubate at room temperature for 10 minutes. Cells were plated to reach 50-60% confluence on the day of transfection. See Girotti M, Banting G. “TGN38-green fluorescent protein hybrid proteins expressed in stably transfected eukaryotic cells provide a tool for the real-time, in vivo study of membrane traffic pathways and suggest a possible role for ratTGN38.” Journal of cell science 109 (Pt 12), 2915-2926 (1996). The cocktail mix was added dropwise to the cells growing in complete medium. The dish was swirled, and then incubated at 37° C. for 36 hours. After this period, cells were washed and allowed to rest for 2 hours before treatment and imaging.

Electron Microscopy. 6-well plates harboring induced cell lines were grown to 80-90% confluence, washed twice with 1×HBSS, and fixed with an acceptable EM fixative. Plates were taken to the CLC Imaging Core Facility at Weill Cornell Medicine for preparation and imaging.

RNA Sequencing (RNAseq). To ensure adequate starting material, two biological replicates each of vector and K-ras^{G12V} were grown in 6-well plates so that three wells were dedicated for each replicate. On the day of extraction, media was aspirated and 500 μL of Trizol LS reagent (Thermo 10296010) was pipetted into each well. Plates were placed on wet ice, and the contents of each well were agitated and scraped with a rubber policeman to ensure all material was homogenous before transfer into a pre-cooled 2 mL Eppendorf (Fisher 05-402-240. Samples were flash frozen in isopropanol/dry ice slurry and stored at −80° C. until processing by the MSKCC Integrated Genomics Operation (IGO): extraction, poly-A enrichment, quality control, library preparation, and sequencing (30-40 million reads, HiSeq-PESO). Downstream bioinformatics was performed by the MSKCC Bioinformatics Core (BIC) using a standard delivery pipeline for alignment, clustering, Htseq counts, and differential expression.

PC-SUV Vesicle and ANS Assay. 500 mg of Soy bean phosphatidylcholine (Lipoid) was dissolved in 200 μL of ethanol; 25 μL of ethanol-lipid solution was injected into 750 μL of deionized water. The resulting MLV dispersion was extruded four times with a manual extruder through a 200 nm membrane. The resulting small unilamellar vesicle (SUV) size was ˜160 nm. See Portnoy E, et al. “Indocyanine Green Liposomes for Diagnosis and Therapeutic Monitoring of Cerebral Malaria.” Theranostics 6, 167-176 (2016). For the ANS assay, a 100 μM stock of 8-anilino-1-naphthalenesulfonic acid ammonium salt (Sigma 10417-F) was dissolved in deionized water. A stock solution of sodium dodecyl sulfate, SDS (Fisher BP166-100), was generated in deionized water. The assay was prepared by first mixing an excess working concentration of 2 μM ANS with 1 μM SUV (calculation based on total lipid) in 1×PBS. To this mixture the vehicle blank, SDS, or reporter was added. As a control, ANS was mixed with SDS or reporter in the absence of SUV. Fluorescence was measured on a Tecan Infinite M1000 Pro plate reader (Tecan Group Ltd.) after a 30-minute benchtop incubation using UV transparent half-well 96 well plates (Corning 3679). The program was set for 350 nm excitation and 470 nm emission (5 nm bandwidth). Top mode was used, with a manual gain of 120 and 50 flashes on mode 1 (400 Hz). Settle time was 0 ms and the z-position was 22303.

Methylene Blue Staining. After cell line induction for 24 hours, a working stain was produced by diluting a 1% w/v stock solution (Fisher 525431) to 0.05% v/v with 1×HBSS. Cells were washed and covered with this working solution, then allowed to incubate for 5 minutes at 37° C. Cells were thoroughly rinsed and maintained in RT fresh 1×HBSS for subsequent imaging. A wide field microscope, color camera, and 10× objective were used for image acquisition.

Retroviral Delivery of shRNA against group 7 genes. RT3GEPIR miR-E vector backbone was used to clone in shRNA targeting sequences (two per gene target) against Renilla luciferase (vector control), pla2g7, or pafah2. shRNA inserts were 22 bases long. Plasmid DNA was generated and supplied by the Gene Editing and Screening Core at MSKCC in part using an algorithm for shRNA generation. See Pelossof R, et al. “Prediction of potent shRNAs with a sequential classification algorithm.” Nature Biotechnology 35, 350-353 (2017). Plasmid amplification was performed using Mach1 competent bacteria inoculated onto 100 mm LB Agar plates (Teknova L111002). Bacterial stocks were first expanded using manufacturer instructions (Zymo Research T3002) followed by storage at −80° C. Transformation of plasmids into bacteria took place on wet ice: 20 μL of competent Mach1 was mixed gently with 250 pg of DNA. This volume was drop pipetted onto 37° C. pre-warmed 100 mm LB Agar plus Carbenicillin (200 μg) plates (Teknova L1046) and was followed by bead rolling. Plates were incubated in a dry 37° C. incubator for 18 hours or until robust colonies formed. Colonies were amplified by inoculating a single transformant colony into 3 mL Terrific Broth plus 200 μg Carbenicillin (Teknova T7510; plus 100 μg Carbenicillin, Fisher AAJ67159AD). Culture tubes with loose fitting caps were incubated 15 hours at 37° C. with shaking (225 rpm). For archiving, 900 μL was taken from the resulting cloudy broth and was mixed 1:1 with 50% sterile glycerol and frozen at −80° C. The remaining broth (or full volume if archiving was not performed) was spun down (6800 rcf, 3 minutes, 15° C.) for mini-prep (Qiagen 27106). For re-amplification of plasmids archived in glycerol, a sterile loop was briefly scraped against the glycerol stock and serial-streaked across two 100 mm LB Agar plates plus Carbenicillin 200 μg. Retroviral packaging was achieved using a Phoenix-AMPHO cell line derived from the HEK293T (ATCC CRL3213). Phoenix was expanded then frozen as aliquots at passage 3 in liquid nitrogen; for use, 2.5E6 Phoenix cells were thawed into basal growth medium as described elsewhere, supplemented with 10% heat inactivated FBS but without antibiotics, onto 60 mm plates 24 hours before transfection to achieve ˜80% confluence. Fresh medium was replaced at 12 hours post seeding. The transfection cocktail made up for each 60 mm dish was: 6 μg plasmid DNA diluted into 1 mL growth medium without FBS/antibiotics followed by a 1:1 ratio DNA:PLUS reagent (ThermoFisher 15338100) and gentle mixing. Mix was incubated at room temperature for 10 minutes followed by the addition of 20 μL Lipofectamine LTX (ThermoFisher 15338100); this mix was incubated for 25 minutes at room temperature. 2 mL of conditioned medium from Phoenix dishes ready for transfection was mixed with 2 mL fresh medium without antibiotics. 25 μM chloroquine was added to this 4 mL volume. This mixed volume was replaced onto Phoenix dishes to be transfected, gently to not disturb the monolayer. The transfection cocktail was then added, dropwise, to the Phoenix dishes, which were allowed to incubate at 37° C. for 9-10 hours. After this time, medium was aspirated and 5 mL of fresh medium supplemented with antibiotics and 10 mg/mL sterile filtered BSA (Sigma A1470 dissolved in 1×PBS) was added gently into each dish. Dishes were replaced into the incubator and, assuming 100% confluence of the Phoenix cells, viral supernatant harvest began at least 24 hours later by pooling the Phoenix conditioned medium representing each gene construct and filtering through a sterile 0.45 μm filter. Due to media acidification, filtered viral supernatant was buffered with 10% v/v HEPES (ThermoFisher 15630106) and 25% v/v fresh complete growth medium. The filtered viral supernatant was snap frozen and stored in sealed conical tubes at −80° C. Target MEF lines were infected with virus by first plating 5E5 target cells per 100 mm dish overnight in complete medium with 5% FBS. Frozen viral supernatant was rapidly but incompletely thawed in a 37° C. water bath. A transduction cocktail was made by diluting 4 mL of viral supernatant with 1 mL fresh medium supplemented with 5% FBS. Polybrene was then added to a final concentration of 4 μg/mL, and the cocktail was gently mixed and incubated at room temperature for 10 minutes. Transduction cocktail was added to aspirated target cell plates. Dishes were incubated at 37° C. for 8 hours, after which fresh 5% FBS supplemented medium was added to bring the dish volume up to 10 mL. Incubation was continued for an additional 36 hours or until cells reached 90% confluence. Conditioned medium was washed off completely using HBSS and complete medium and then target cells were split into new dishes to achieve 80-90% confluence two days later for the start of dual antibiotic selection. To select, complete growth medium was supplemented with 30 μg/mL hygromycin for maintenance of the original pTURN vector (killing concentration ˜150 μg/mL) and 3 μg/mL puromycin for selection of cells harboring the shRNA constructs. Uninfected and untreated controls were grown in parallel. Complete medium with antibiotics was replaced on target cells every two days until the control culture died. Cell colonies proliferating under selection were expanded, exposed to a full hygromycin selection dose for two additional days to ensure presence of the original inducible cDNA, and then harvested for storage or immunoblot analysis. Assays showed that the appropriate lines (iKRas/mirG7 or iKRas/mirAH2 versus Renilla control) overexpressed RasG12V and had reduced levels of group 7 or pafah2 protein; however, the control lines showed elevated inflammatory lipid contents when assayed by nanosensor and showed elevated PAFAH activity when assayed with a kit. iKRas harboring knockdown lines showed no distinct change in nanosensor response and assay of PAFAH reported elevated activity in cell lysate and conditioned culture medium (data not shown). For these reasons, stable inducible shRNA was not reported and instead transient RNAi was used.

Immunocytochemistry for Differentiation Markers. The following primary antibodies were used for indirect immunofluorescence: 0.1 μg/mL anti-vimentin mouse monoclonal (Vector Labs VPV684), 1 μg/mL anti-SMA mouse monoclonal (Sigma A5228), 2.5 μg/mL anti-E-cadherin mouse monoclonal (BD 610181), 1:300 anti-Syntaxin 6 (Cell Signaling 2869), 1:300 anti-LAMP1 (Abcam 25245). Results showed that iKRas did not express any of these terminal differentiation markers when compared to background staining in vector or to a positive control cell line, HK-2 for E-cad (data not shown).

Reagents and Materials. Nano-reporter was prepared as described;⁸⁸ in brief, raw nanotube (SWCNT) was suspended with single-stranded DNA (ssDNA) sequence 5′-CTTCCCTTC-3′ (IDT Technologies) and subjected to Aqueous Two Phase (ATP) separation to purify the (9,4) chirality. Stock solutions were kept at 4° C. For confocal imaging, 1 μmole of Alexa647-ssDNA (5′-CTTCCCTTCTT/iSp18//3AlexF647N/-3′, IDT Technologies) was used to generate the reporter. Dispersion of 1 mg raw SWCNT (NanoIntegris, HiPco) with 1 mg ssDNA dissolved in 0.1 M NaCl was achieved by sonicating (2 mm stepped probe, Sonics and Materials Inc., Pulse: 1-minute ON, 15 seconds OFF) the mixture for 30 minutes in a −20° C. cold block. The resulting suspension was benchtop centrifuged for 10 minutes at 30,000 rcf. The supernatant was ultra-centrifuged for 30 minutes at 171,180 rcf. The resulting supernatant was spin-filtered (Millipore Amicon, 100 kDa) three times against deionized water, re-suspended, and then centrifuged for 10 minutes, 30,000 rcf. The top 90% of the supernatant was collected and measured by UV-Vis-NIR spectrophotometry (Jasco V-670). The extinction coefficient used to calculate concentration was: c (910 nm)=0.02254 L·mg⁻¹·cm⁻¹. PBS and HBSS were prepared by the Memorial Sloan Kettering Media Preparation Core Facility. DMSO (Fluka BP2311) was stored with molecular sieves (Fluka 69839). Drugs and compounds used in this work were as follows: Doxycycline hydrochloride solution (Sigma D3072), Varespladib (Selleckchem S1110), MJ-33 (Cayman 90001844), BEL (Cayman 70700), MAFP (Cayman 70660), Darapladib (Selleckchem S7520), Rilapladib (MCE HY-102004/CS-0022446), ML256 (gift), AA39-2 (gift), P11 (Cayman 17507), TSI-01 (Cayman 17628), methylcarbamyl PAF C-16 (Cayman 60908), (±)-α-Tocopherol (Sigma T3251), phosphocholine chloride calcium salt tetrahydrate (Sigma P0378), linoleic acid (Sigma L1376), 1-C16 ether MG (Avanti 999971), 18:1 BMP (S,R) (Avanti 857133), C18 LPA (Avanti 857228), 18:1 sphingosine-1-phosphate (Avanti 860492), 18:0 PA (Avanti 830865), 8:0 LPC (Avanti 855275), 14:0 LPC (Avanti 855575), 16:0 LPC (Avanti 855675), C16 2:0 PAF (Avanti 878110), 18:0 LPC (Avanti 855775), 18:1 LPC (Avanti 845875), 20:0 LPC (Avanti 855777), 24:0 LPC (Avanti 855800), 14:0 PC (Avanti 850345), 16:0 PC (Avanti 850355), 18:0 PC (Avanti 850365), 16:0 LPS (Avanti 858142), 16:0 PS (Avanti 840037), 18:0 LPS (Avanti 858144), 14:0 LPE (Avanti 856735), 18:0 LPE (Avanti 856715), 14:0 PE (Avanti 850745), 16:0 LPG (Avanti 858122), 16:0 PG (Avanti 840455), 16:0 LPI (Avanti 850102), 16:0 PI (Avanti 850141), NaCl (Fisher S2711), NP-40 (Sigma 18896), sodium dodecyl sulfate (SDS) (Sigma 436143), sodium deoxycholate (SDC) (Sigma D6750), EDTA (Sigma EDS); TAK-632 (500 nM; Selleckchem S7219), GDC-0941 (500 nM; Selleckchem S1065), Torin1 (500 nM; Selleckchem S2827), Trametinib (500 nM; Selleckchem S2673), Losmapimod (500 nM; Selleckchem S7215), JNK-IN-8 (20 nM; Selleckchem S4901), GDC-0994 (500 nM; Selleckchem S7544), dexamethasone acetate (Cayman 22286), BQU57 (Selleckchem S7607), QNZ(EVP4593) (Selleckchem S4902), leupeptin (Selleckchem S7380), pepstatin (Selleckchem S7381), E-64 (Selleckchem S7379), 16:0 LPC (Avanti 855675), Darapladib (Selleckchem S7520), AA39-2 (gift), LPDS (Sigma S5394), delipidated BSA (Fraction V fatty acid free BSA, Sigma A7030, Lot #5LBQ0873V), docosahexanoic acid (DHA) (Sigma D2534), linoleic acid (LA) (Sigma L1376), and linolenic acid (ALA) (Sigma L2376).

In vivo survival study. To generate the syngeneic KP transplant lung cancer model, a cell line derived from a KrasG12D/+; Trp53−/−(KP) GEMM lung tumor was infected with a retroviral luciferase (Luc)-GFP construct, and 5000 KP tumor cells resuspended in 400 μL of PBS were tail vein injected into 7 week old Female C57BL/6 mice.⁹⁰ Mice were randomized into various study cohorts before treatment with vehicle or darapladib. Treatments were administered by oral gavage once per day, Monday through Friday. Mice that did not die with disease-related morbidities by Day 73 post-xenograft were monitored for tumor formation by bioluminescence imaging (BLI) on a Xenogen IVIS Spectrum (Caliper Life Sciences) and their weight changes were evaluated. Bioluminescence positive mice remained in the study while bioluminescence negative mice were euthanized, and their lungs were harvested for histology. Tissues were rinsed and fixed in 4° C. buffered 4% paraformaldehyde for 24-48 hours and moved to 4° C. 70% ethanol for long term storage, embedding, sectioning, and staining (H&E, anti-GFP primary antibody, anti-Ki-67 primary antibody). Due to the possibility that some mice did not successfully seed with tumor cells from the injected volume, mice from the Day 73 survivor group were harvested and evaluated by histology. Tissues negative for histological dysplasia were excluded from final study analysis; tissues positive for histological dysplasia were kept in the final study analysis. Each cohort's N value was adjusted to reflect histology results before final analysis. One treatment cohort mouse negative for bioluminescence was labelled as an outlier in the final analysis due to a very early death (Day 24) without disease-related morbidity, and one treatment cohort mouse positive for lung bioluminescence was euthanized once its weight dropped but before disease-related death (Day 91) to keep a reasonable study timeline.

Human expression analysis. Human data from TCGA PanCancer Atlas Studies⁹¹ was accessed via the cBioPortal.⁹² A query of pla2g7, pafah2, kras, and hras genes was conducted. Output data is RNAseq V2, ranked by median expression, either linear or log 2 scaled.

Results

Oncogenic RAS Amplification Induces Senescence-Associated Stress and Damage.

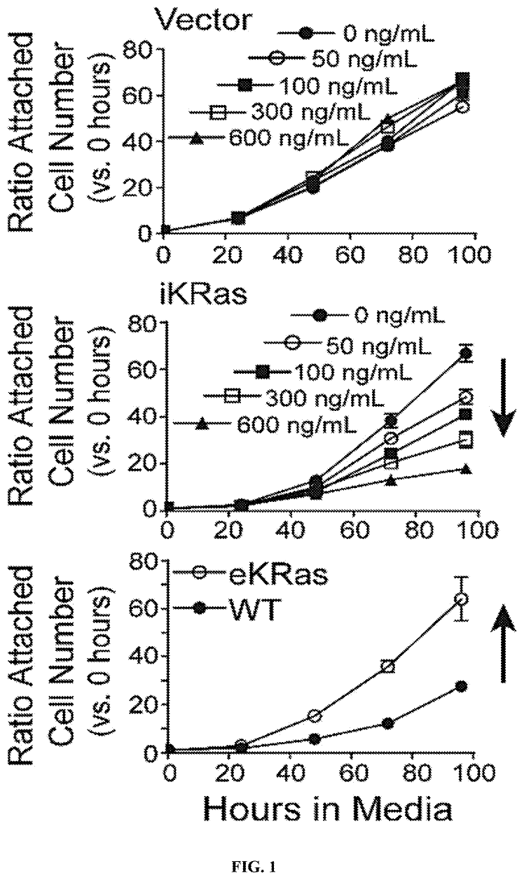

The extent of RAS stress and damage in SV40 large T-immortalized murine embryo fibroblasts stably transduced by Tet-inducible K-or-H-ras^{G12V} cDNA (iK/HRas) was assessed. First measured was the proliferation of iKRas, iHRas, or vector (control) MEFs exposed to doxycycline (FIG. 1). For comparison, endogenous oncogenic K-ras (eKRas) cells generated from embryos pre-treated by Cre recombinase were immortalized and tested for proliferation alongside paired wildtype MEFs. There was a clear difference in proliferation between the vector controls, the reversible Tet-ON lines, and the endogenous mutant. While KRAS amplification arrested cells, endogenous KRAS promoted hyperproliferation.

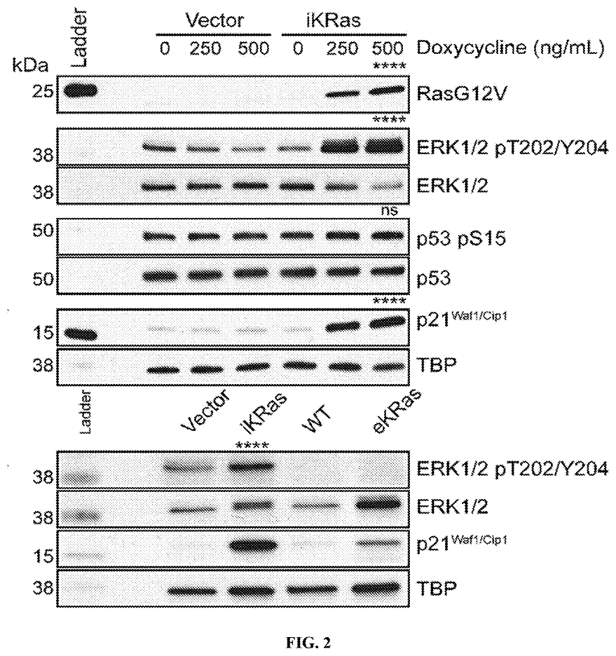

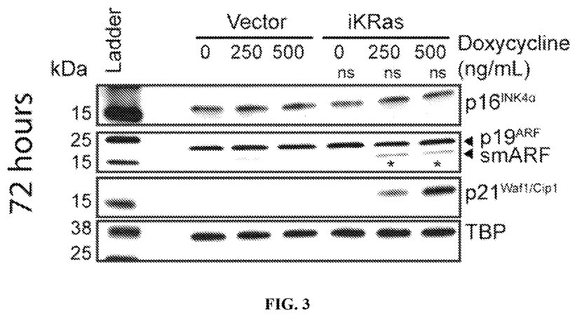

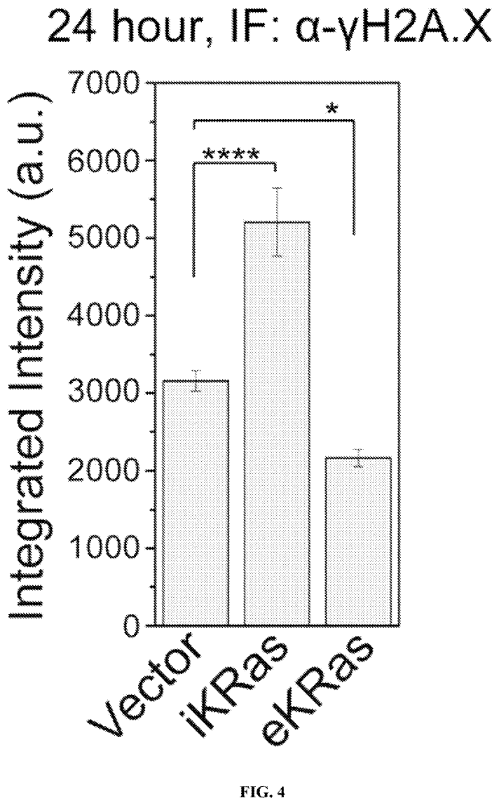

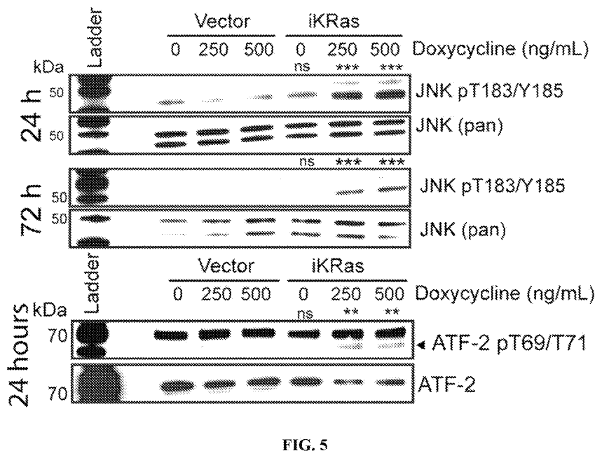

To investigate the signaling changes in these two models, a 24 h immunoblot was performed on cell lysates (FIG. 2). The immunoblot showed oncogenic RAS over-expression and downstream ERK1/2 phosphorylation in iKRas cells. Notably, phospho-p53 showed no significant increase; however, p21^{waf1/cip1} expression was elevated and maintained expression throughout doxycycline treatment (FIG. 3). Compared to the amplified RAS cells, in eKRas endogenous RAS cells the ERK phosphorylation was not detectible and p21^{waf1/cip1} was barely detectible. To determine whether other key attributes of oncogenic RAS stress were present, a DNA-damage response was tested. FIG. 4 shows the quantification of immunofluorescence from an acute double-strand break marker, γH2A.X. Similarly, an assay for the genotoxic stress markers, phospho-JNK and phospho-ATF-2, was performed (FIG. 5). All stress markers were elevated in iKRas cells, but not eKRas or control.

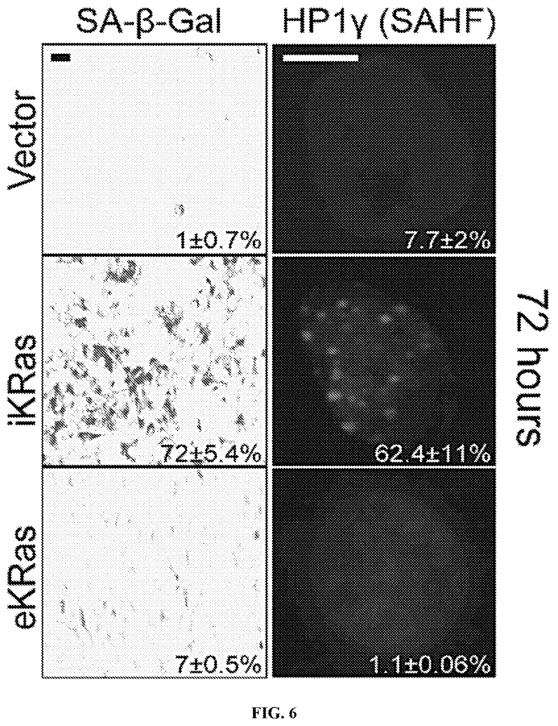

To confirm the relationship between oncogenic RAS dosage and pre-mature senescence, the inventors assessed whether RAS mutant cells exhibited a difference in senescence markers. Keeping with a two-marker minimum for stress-induced senescence, the inventors assayed for senescence-associated β-galactosidase and heterochromatin foci (HP1γ). Both of these assays reported elevated senescence-associated marker expression only in iKRas cells after 72 h in culture (FIG. 6). In the case of the eKRas cells, β-Gal signal was stochastic. Given the clear oncogenic-stress phenotype expressed by iKRas cells, the inventors inquired whether they would generate an acute SIR/SASP inflammatory expression profile. RNA sequencing of 24 h induced cell cultures, as well as immunoblots for representative SIR/SASP markers reported the upregulation of an innate inflammatory response. Based on previous findings of SIR and SASP phenotypes from long-term cell culture,^{23, 28, 34, 35, 36, 37} our profiling indicated that iKRas, but not eKRas cells, modeled a stress and damage response to amplified oncogenic RAS.

Oncogenic RAS Stress Triggers Group 7 sPLA2 Activity.

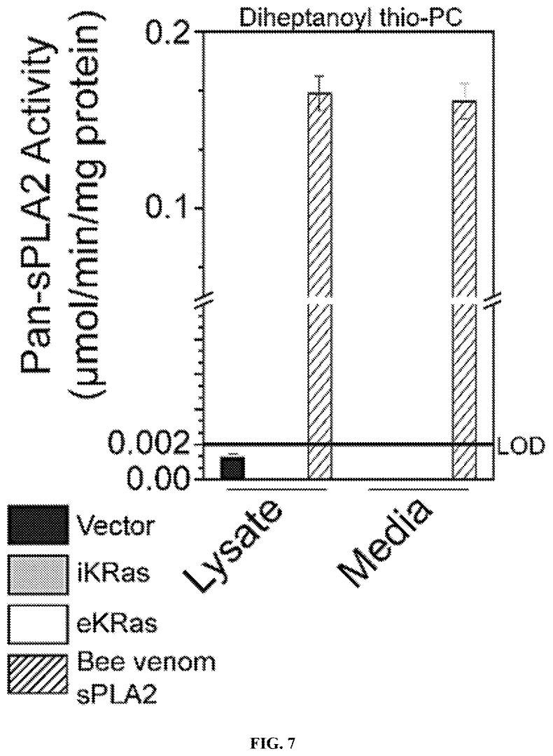

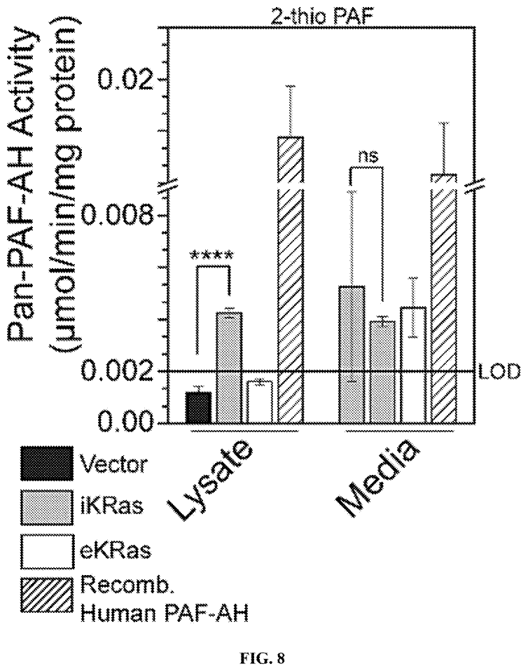

The activity of sPLA2 isoforms in iKRas and eKRas cells was investigated. Chromogenic enzyme assays were run using two lipid substrates: diheptanoyl-phosphatidylcholine (PC), a substrate of most cancer-associated sPLA2 isoforms known to-date, or platelet activating factor (PAF), a substrate of group 7 and 8 sPLA2 isoforms (also known as platelet activating factor acetylhydrolases, PAF-AHs). Cell lysate and conditioned media from the amplified iKRas model, vector control cells, or hyperplastic eKRas were prepared and incubated with either thiolated PC or PAF, mixed with DNTB (Ellman's Reagent). Purified enzymes, bee venom-derived group 3 sPLA2 (of which PC is a substrate), or human PAF-AH (of which PAF is a substrate), were spiked into samples as positive assay controls. No PC-specific activity in iKRas cells was observed (FIG. 7), but elevated PAF-AH-specific activity was observed; neither control nor eKRas lysate contained activity above the limit of detection (FIG. 8). These results evidenced that intracellular group 7 and/or 8 sPLA2 isoforms were induced by amplified RAS-induced damage.

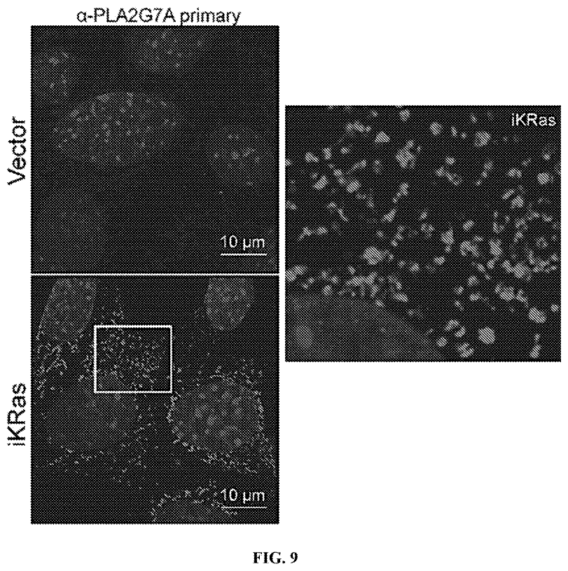

To assess whether intracellular protein expression was consistent with measured group 7 sPLA2 activity, confocal microscopy was conducted and mRNA sequence data was analyzed. Immunocytochemistry using anti-PLA2 group 7A antibody showed that iKRas cells contained endogenous overexpression and punctate (vesicular) localization of the 7A isoform (FIG. 9). It was found that the sPLA2 group 7B isoform, which relocates from cytosol to ER/Golgi during stress,³⁸ stained diffusely and with low intensity in iKRas cells. These results evidence that group 7B was not the major source of intracellular PAF-AH activity in iKRas cells. Furthermore, results were consistent with a 32-fold amplification of pla2g7 RNA. Group 7B and group 8 enzymes were not similarly over-expressed in iKRas cells.

Lipid Dysregulation in iKRas Endomembrane is Due to PLA2G7 Activity and Oxidized Phospholipid.

Because group 7A sPLA2 is a normal component of animal serum lipoproteins, the inventors surmised that iKRas cells may exhibit dysregulated intracellular lipid metabolism related to the endogenous activity of this circulatory enzyme. Lipid dysregulation in cellular endosomal organelles was assessed using a previously-validated nanosensor that measures soluble lipids by a shift in its near infrared emission towards smaller (bluer) values in the endosomal lumen of live cells.^{39, 40} The inventors first queried whether the sensors would respond to the substrates/products of sPLA2 enzymes.³⁹ The reporter was confirmed to be pH-insensitive and membrane bilayer-impermeable. First interrogated was the sensor response in vitro to individual lipids that represent a wide-range of endogenous surfactant-class (water-soluble) species, where it was found that the chromatic response of the reporter was most sensitive to lysophosphatidylcholines (lysoPC), platelet activating factor (PAF), and lysophosphatidylserine (lysoPS). Additional sensitivities, e.g. to very-long chain lysoPC and PC, were observed at lipid concentrations far above their endogenous critical micelle concentrations (pM-nM, above this point free solubility is negligible).

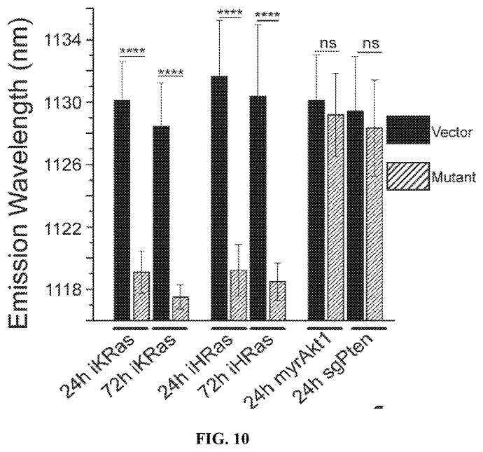

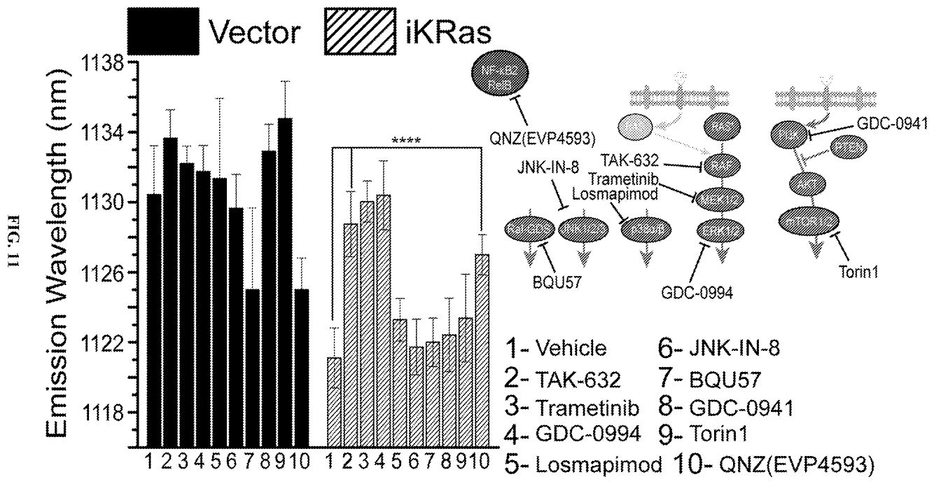

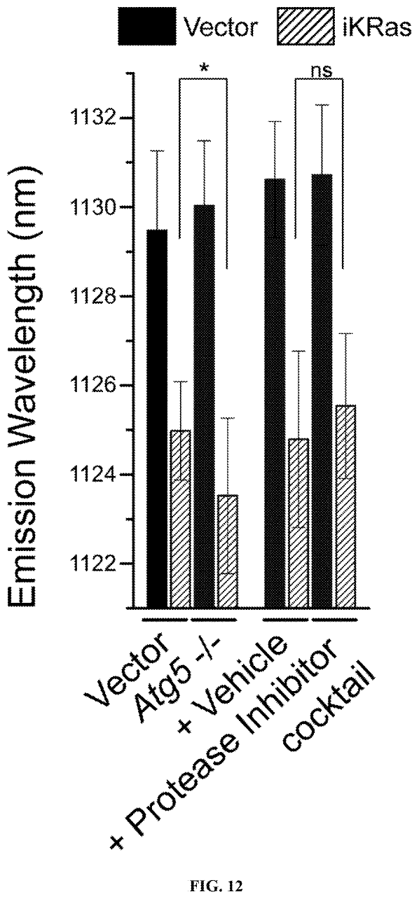

To assess lipid dysregulation in live cells, the lipid nanosensor was introduced into the culture medium of RAS- or vector-transformed cell lines. Near-infrared hyperspectral microscopy was utilized to acquire the fluorescence spectra of the nanosensor spatially within iKRas, iHRas, eKRas, vector control cells, and MEFs harboring mutations in the parallel PI3K pathway. The data were fitted to determine the center wavelength of the nanosensor emission band, which was then mapped back over the brightfield images of the cells and plotted in a histogram. Using this method, the average sensor emission wavelength 24 h and 72 h after oncogenic RAS induction was measured. The average sensor emission wavelength 24 h after culturing immortalized MEFs harboring myristoylated (constitutive) Akt1 (myrAkt1) or short guide RNA to knockout Pten was also measured (FIG. 10). It was found that only the constitutive RAS mutants exhibited significantly blue-shifted responses, as compared to either vector or signaling control cells. eKRas cells exhibited a minor blue-shift that was significantly less than the shift in iKRas cells. Pharmacologic inhibitors of MAPK pathway effectors, such as RAF, MEK, and ERK, (TAK-632, trametinib, and GDC-0994, respectively) significantly diminished the lipid nanosensor response in iKRas relative to vector, as compared to pharmacologic inhibitors of mTOR/AKT pathway effectors, which did not affect sensor response (FIG. 11). Inhibitors of vesicular degradation pathways similarly did not abrogate the nanosensor response (FIG. 12). These results indicate that damage from oncogenic RAS amplification directly induces endosome/endomembrane lipid dysregulation that is not particular to any one endomembrane trafficking pathway.

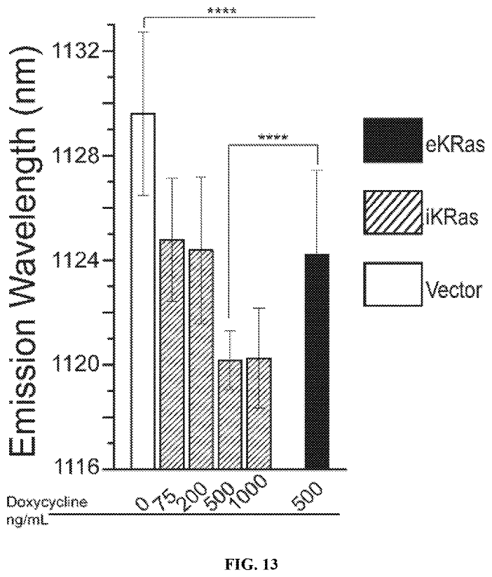

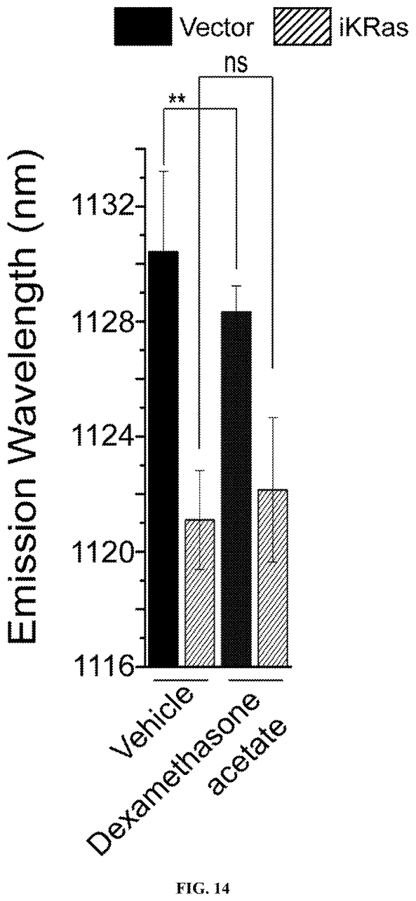

Next measured was whether this intracellular reporter response was sensitive to sPLA2-mediated generation of lysophospholipids and related lipid species. Lipid accumulation was measured using the nanosensor 24 h after treating the cells with an array of PLA2 inhibitors. The nanosensor response showed that the lipid dysregulation in iKRas cells was prevented only by inhibitors of group 7 sPLA2 (PLA2G7A/B) enzyme activity (FIG. 13). The inhibitors attenuated the reporter response in the following order: darapladib>rilapladib≈ML256>AA39-2. Darapladib and rilapladib are potent and reversible PLA2G7A inhibitors, while ML256 is a covalent inhibitor of PLA2G7A and AA39-2 is a covalent inhibitor of PLA2G7B.^{41, 42} The other PLA2 inhibitors that were unable to attenuate the lipid reporter response in iKRas cells included varespladib, which inhibits PLA2G2/5/10/12 isoforms, MJ-33, a transition state analog of arachidonate⁴³ that reportedly inactivates PRDX6 and PLA2G15, BEL, an inhibitor of PLA2G6, MAFP, an inhibitor of cytosolic PLA2G4, P11, an inhibitor of PLA2G8, and TSI-01, an inhibitor of PAF biosynthesis. Consistent with group 7 activity in RAS-damaged cells, the PLA2G7A stimulator and anti-inflammatory drug, dexamethasone,⁴⁴ increased the reporter response in the vector line (FIG. 14). These results evidence that group 7 sPLA2 (PLA2G7A/B) activity, but not broad sPLA2 activity, induces endomembrane lipid dysregulation, and damage from oncogenic RAS amplification markedly exacerbates this dysregulation.

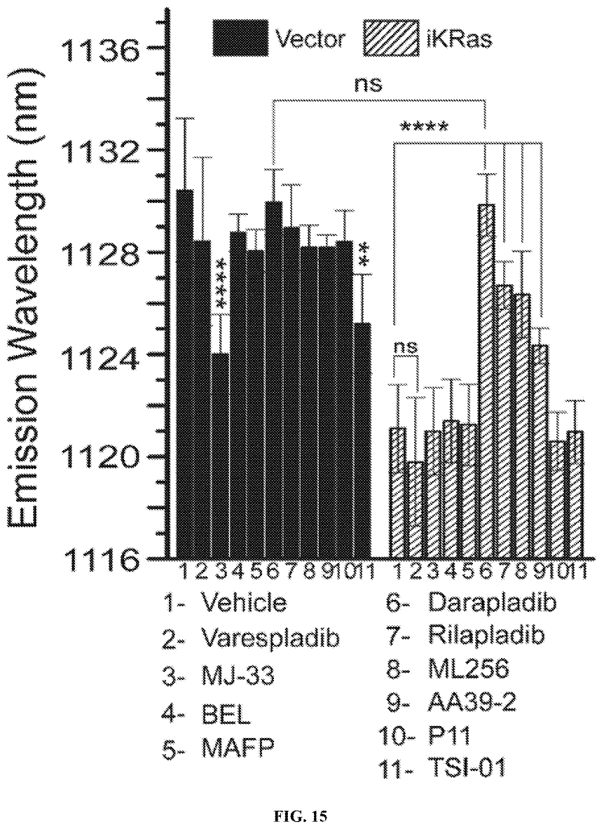

Also examined was whether iKRas lipid dysregulation was potentially caused by membrane phospholipid oxidation. First, to assess the oxidizing environment of iKRas cells, live cell cultures were stained with the redox dye methylene blue, which showed elevated cellular staining. This result supports an elevated intracellular oxidizing environment. iKRas cells were then incubated with lipophilic α-tocopherol (Vitamin E), a common membrane antioxidant.⁴⁵ The reporter response was abrogated in cells pre-incubated with Vitamin E (FIG. 15), suggesting that the lipid dysregulation resulted from oxidative damage to membranes to result in the release of soluble lysophospholipid.

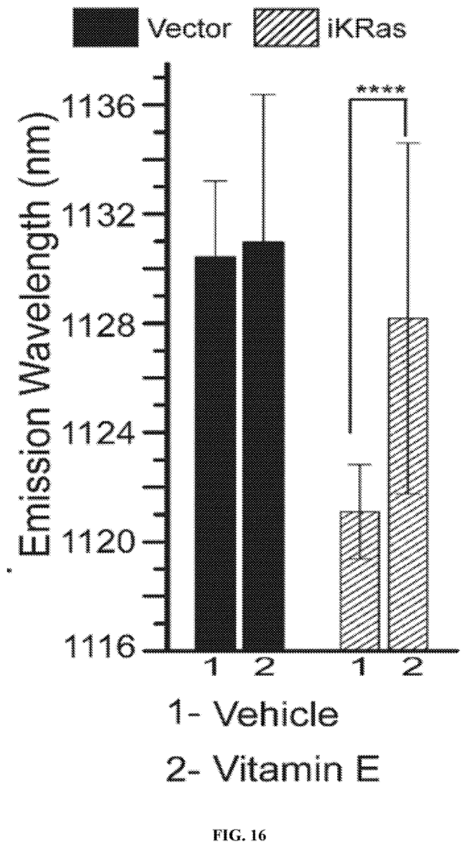

Next investigated was whether the endosomal lipid dysregulation required the polyunsaturated fatty acid (PUFA) components of serum lipoproteins, which are necessary for the synthesis of group 7 substrates PAF′ and oxidized phospholipids (PAF-analogs). Cells were pre-incubated in lipoprotein-deficient serum with or without two essential PUFAs: arachidonic acid (AA) and docosahexaenoic acid (DHA). It was observed that the lipid reporter response was abrogated upon PUFA depletion but rescued after PUFA supplementation (FIG. 16), indicating that this lipid dysregulation also required the polyunsaturated fatty acid substrates of group 7 sPLA2 enzymes.

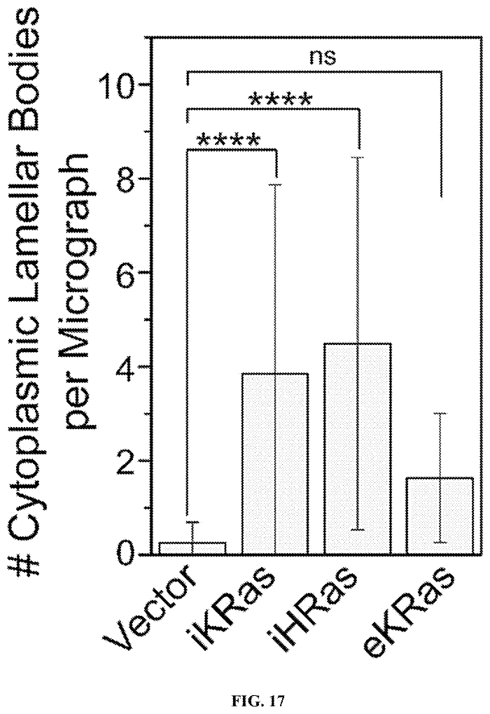

Electron microscopy of oncogenic RAS-amplified cells were conducted to further investigate the lipid dysregulation phenotype. Transmission electron microscopy (TEM) showed the presence of unusual lamellar-like structures in iKRas and iHRas lines (FIG. 17). These structures appear to resemble lamellar bodies.⁴⁷ These images further support lipid dysregulation within the lumen of endosomal and/or secretory endomembrane compartments and these images also support intracellular retention of PLA2G7 as an important component of oncogenic RAS-induced damage.

Based on the reporter data presented heretofore, a working model was tested wherein direct damage to the endomembrane increases the production of PAF-analog substrates and lysophospholipids through the action of group 7 sPLA2 enzymes.

RAS Amplification-Mediated Lipid Oxidation Enhances Cellular Lysophospholipids Through PLA2G7.

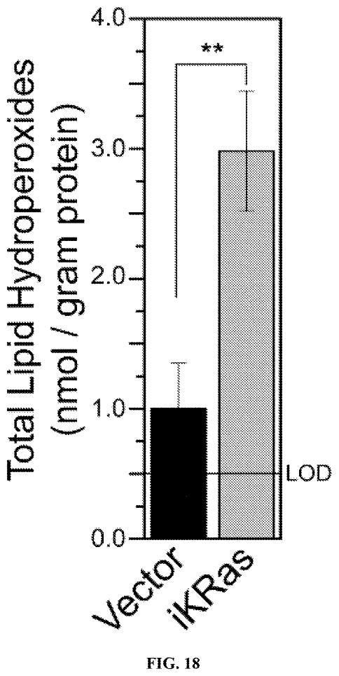

To address the working model, it was investigated whether the lipid species produced by iKRas cells were consistent with activity from a group 7 sPLA2 enzyme. A total membrane lipid hydroperoxide chromogenic assay was performed on iKRas to test for the presence of group 7 sPLA2 enzyme substrates. The assay (FIG. 18) showed that iKRas cells contained elevated lipid hydroperoxide levels over the control, supporting increased production of group 7 enzyme substrate.

To determine whether iKRas metabolism produced a lipid signature consistent with group 7 sPLA2 activity, iKRas cell lysates and conditioned media were prepared for LC-MS/MS of glycerophospholipids, sterols, glycerolipids, and sphingolipids. It was found that lysoPAF and lysoPC species were enriched in the conditioned media of the mutant cultures, as compared to the vector control. The iKRas cell lysates contained less total saturated/unsaturated phospholipids, plasmalogen phospholipids, and ether phospholipids, but they contained more lysophospholipids than the vector control. These results support the conclusion that increased lipid dysregulation includes sPLA2 products.

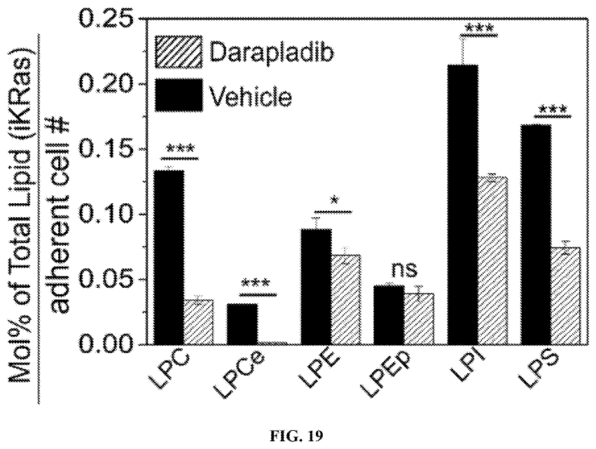

The inventors reasoned that the loss of saturated/unsaturated phospholipids and a rise in lysophospholipids in iKRas cells could be due to direct oxidative modification, enzymatic action, or both activities. To understand whether group 7 sPLA2 activity did indeed contribute to phospholipid dysregulation, lipid metabolism was assessed after iKRas cells were treated with the group 7 enzyme inhibitor, darapladib. Lipidomics data showed significant attenuation of lysophospholipid levels, especially lysoPC, but also other PUFA-harboring classes like lysoPE, lysoPI, and lysoPS (FIG. 19), suggesting that group 7 enzyme activity was at least partly responsible for the lysophospholipid generation in iKRas. Additional lipidomic analysis found a general loss of lipid species in iKRas cell lysate and media, evidencing that the iKRas phenotype was broadly catabolic and/or that damage and modification of lipids was not unique to the phospholipid class.

Group 7 sPLA2 Substrate and Product Lipids Differentially Affect Cell Proliferation.

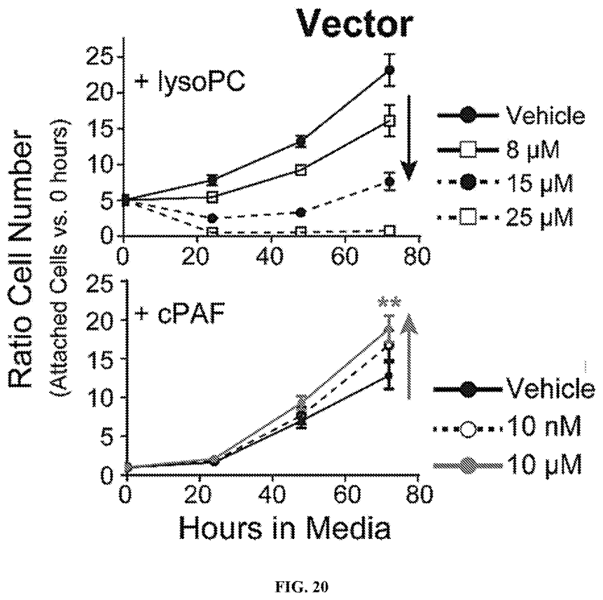

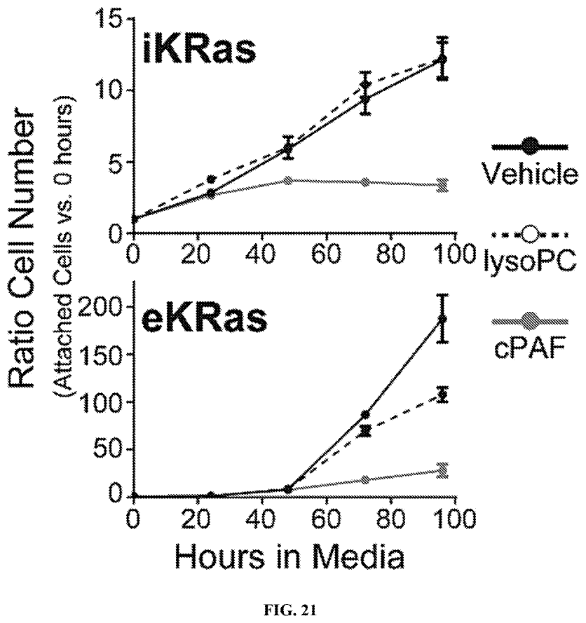

As sPLA2 enzyme isoforms have been described as both tumor suppressors and tumor promoters,^{48, 49, 50, 51, 52, 53} iKRas cells were interrogated using two representative lipids—a group 7 sPLA2 substrate, and a product. First, the vector control cells were incubated with increasing concentrations of 16:0 lysoPC, a group 7 sPLA2 product, and 16:0 methylcarbamyl PAF (cPAF), a substrate. It was found that the lysoPC product arrested vector control cell proliferation (FIG. 20) near the lysoPC critical micelle concentration (CMC, ca. 8-10 μM). The arrest at this concentration suggested a direct detergent-like effect. On the other hand, the group 7 sPLA2 substrate, cPAF, exhibited a mild stimulatory effect on control cell proliferation at both sub-critical and critical micelle concentrations (CMC similar to lysoPC). Therefore, in the vector control cells, intracellular accumulation of the group 7 sPLA2 product affected proliferation to a greater extent than the enzyme substrate. On interrogating iKRas and eKRas cells with critical micelle concentrations of the same lipids, lysoPC promoted either a negligible arresting effect or a reduced arresting effect, while cPAF promoted a potent arresting effect on both iKRas and eKRas cells (FIG. 21). Therefore, the data from these studies support that intracellular accumulation of the group 7 substrate is significantly more cytostatic than the product lipid. In addition, eKRas cells were much more sensitive to the substrate lipid absent significant RAS-related damage or group 7 activity.

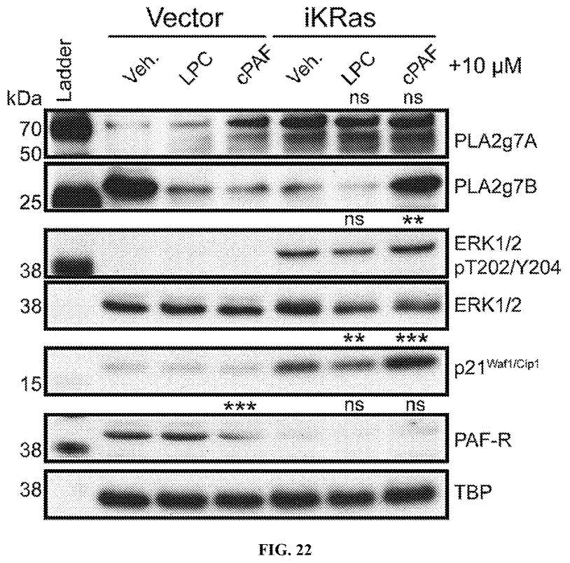

To investigate a molecular link between group 7 and RAS-mediated cellular arrest, it was assessed whether cell membrane loading of the group 7 substrate/product would affect endogenous p21^{waf1/cip1}. Vector and iKRas cells were incubated with lysoPC or cPAF lipids for 24 h. Immunoblots showed that phospho-ERK and p21^{waf1/cip1} expression increased on exposure to cPAF, the PLA2G7 substrate, but not the lysoPC product (FIG. 22), supporting that the accumulation of this lipid substrate is involved in oncogenic RAS-induced arrest.

To assess whether this lipid substrate-related arrest was stimulated endogenously through oncogenic RAS amplification, the inventors immunoblotted for the PAF receptor (PAF-R), an intracellular target that is downregulated in the presence of PAF analogs. It was found that PAF-R was downregulated in vector cells exposed to cPAF, while PAF-R status in iKRas cells was independent of lipid loading (FIG. 22). Because cellular loading of a group 7 substrate triggered the arrest observed after amplification of oncogenic RAS, the data supports that RAS damage itself generates these cytostatic/cytotoxic lipid species.

Group 7 sPLA2 Knockdown Kills Oncogenic KRAS-Harboring Cells.

To assess whether a function of group 7 sPLA2 is to remove deleterious PAF-analog lipid species, the inventors assessed whether knockdown of group 7 would modulate p21^{waf1/cip1} expression. Commercial siRNAs targeting the group 7 sPLA2 genes were incubated with iKRas cells. The inventors immunoblotted lysates to assess the impact of genetic knockdown on arrest, where it was found that phospho-ERK and p21^{waf1/cip1} expression disappeared, supporting that oncogene-induced arrest required group 7 expression. However, removal of group 7 enzymes should permit greater accumulation of damage-inducing PAF analogs, an expectation seemingly inconsistent with the finding that cPAF lipid substrate alone promotes phospho-ERK and p21^{waf1/cip1} expression.

Observations made in the course of this work suggested to the inventors that the iKRas phenotype depended on serum lot and cellular PUFA loading. To determine whether phospho-ERK and p21^{waf1/cip1} expression depended on PUFA, the inventors repeated the above experiment after pooling together the siRNA pairs and supplementing the culture medium with a 15 μM PUFA mix (5 μM each of arachidonic, docosahexaenoic, and linoleic/linolenic acids). The resulting immunoblots showed that phospho-ERK/p21^{waf1/cip1} expression persisted after knockdown in the presence of added PUFA. Similarly, pharmacological blockade of PLA2G7 isoforms in the presence of PUFA increased phospho-ERK/p21^{waf1/cip1} expression, consistent with knockdown of these enzymes allowing the substrate to accumulate intracellularly. The importance of PUFA loading on this enzyme pathway was deduced from the dependence of group 7 sPLA2 protein expression on the presence of PUFA. Therefore, the presence of PUFA-derived substrate was concluded to be an important upstream mediator of both enzyme induction and damage-mediated arrest.

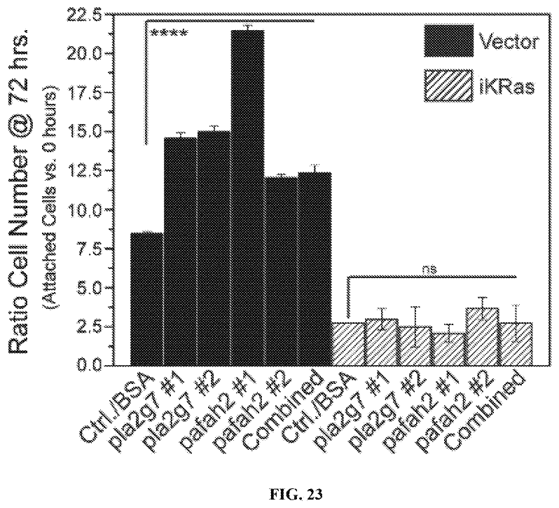

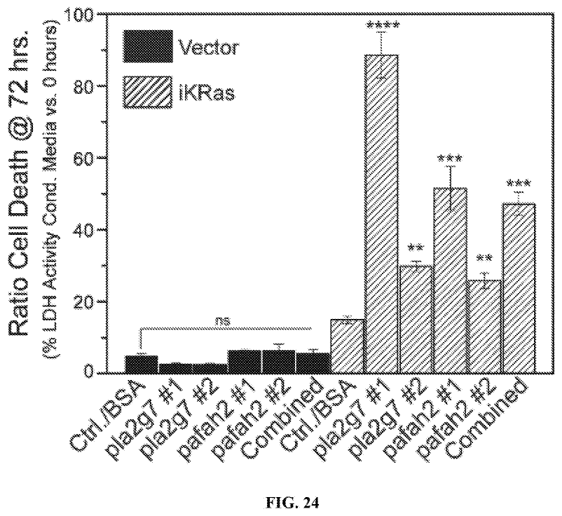

Because group 7 sPLA2 enzymes clear away cytostatic/cytotoxic phospholipids, the inventors assessed whether group 7 knockdown would affect cell proliferation and survival. As before, cells were maintained in culture medium supplemented with PUFAs and siRNAs. Attached cell numbers were counted at 72 h after doxycycline treatment. It was found that group 7 knockdown stimulated vector control cell proliferation, but not iKRas cell proliferation (FIG. 23). To assess cell death, the culture media was removed at 72 h and tested for cell rupture-related lactate dehydrogenase (LDH) activity (FIG. 24). Knockdown resulted in increased LDH activity in iKRas cells but not in the vector control cells, denoting an increase in cell death in iKRas cultures.

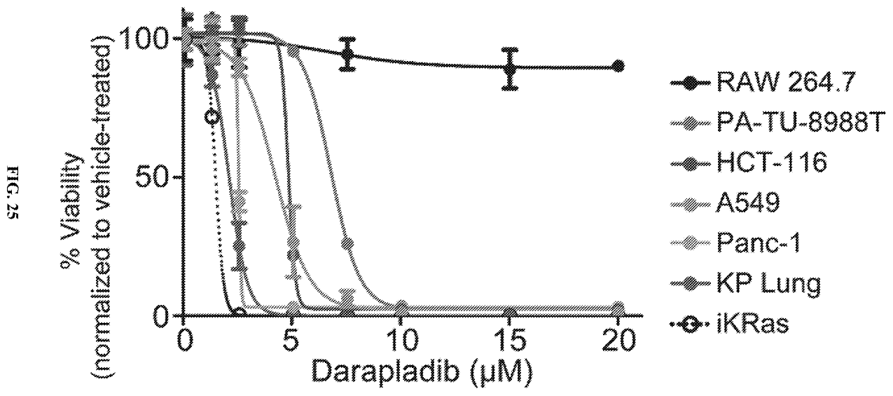

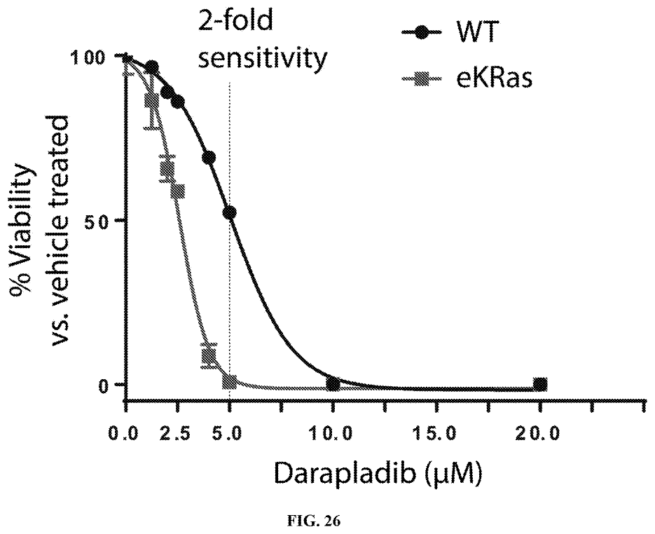

To assess the response of oncogenic RAS-harboring cancer cells to the most developed pharmacologic inhibitor of group 7 sPLA2, several tumor-derived cell lines were interrogated with darapladib, a drug originally developed to treat cardiovascular disease. The iKRas cells and cell lines derived from lung, pancreas, and colorectal tumors were incubated with darapladib and assayed for viability (FIG. 25), where all RAS harboring lines died at low micromolar concentrations darapladib. A control cell line that naturally expresses high levels of group 7 sPLA2, RAW 264.7 murine macrophages, was largely unaffected by the treatment. Because RAW 264.7 cells express group 7 sPLA2 enzymes, these results suggest that the presence of significant membrane damage underlies drug sensitivity. To determine whether darapladib could selectively kill RAS mutant cells, eKRas (having minor oncogenic stress and no significant 7A isoform) or wildtype SV40 MEFs were exposed to the drug. Viability data showed that darapladib was twice as potent at killing eKRas versus wildtype cells (FIG. 26).

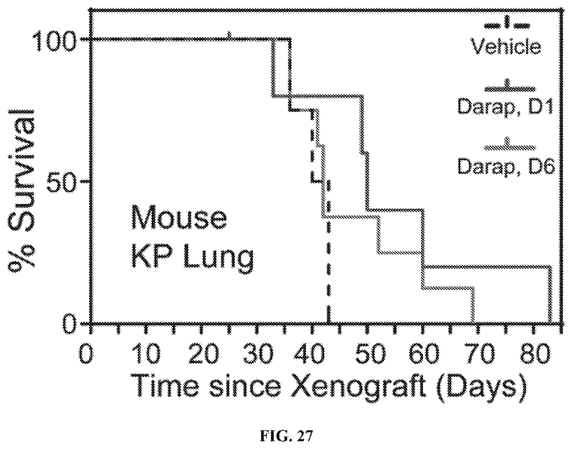

The inventors next assessed whether group 7 inhibition would impact the development of a RAS-harboring murine cancer model. A syngeneic KrasG12D/+; Trp53−/− (KP) transplant model of non-small cell lung cancer was used.^{54, 55} Luciferized cells derived from a GEMM lung tumor were administered intravenously into WT C57BL/6 mice. Mice were treated with vehicle or darapladib by oral gavage starting at two different timepoints after the initial xenograft injection to assess the effect of treatment on cancer development. A significant delay was observed in both lung cancer-associated death and morbidity of mice treated with darapladib (FIG. 27). In addition, the inventors observed non-primary tumors in other organs of the vehicle-treated, but not darapladib, cohort.

Finally, to investigate the tissue and human relevance of this target across cancers, the inventors interrogated the TCGA database for expression information on the two group 7 isoforms and analyzed retrospective data from two studies of pancreatic cancer. The inventors found that group 7 transcripts are expressed and amplified in a wide variety of human cancers, including pancreatic cancer. Almost all these cancers show overexpression and amplification of kras and hras transcripts, including tissues that frequently harbor the point-mutated and constitutive form of RAS.

Discussion

In this Examples section, inflammation-related lipid dysregulation that contributes to cell survival in RAS-mutant tumors was investigated. While various sPLA2 isoforms have been discovered in malignant cells and tissues that harbor oncogenic ras,^{48, 49, 50, 51, 52, 53} a direct relationship between these inflammatory mediators and RAS is a nascent area of research in non-leukocyte models. In an embryo cell line model, the inventors found markedly elevated p21^{waf1/cip1}, pre-mature senescence markers, as well as markers of DNA damage and stress (γH2A.X, phospho-JNK, and phospho-ATF-2) that in total support the link between gene amplification and in vivo carcinogenicity from oncogenic RAS in the developing mouse.^{19, 20, 21, 22} Coupled with the upregulation of senescence markers, SIR/SASP gene and protein upregulation indicated that iKRas cells modelled an inflammatory phenotype.^{23, 28, 35, 56, 57} Notably, eKRas, the single-allele hyperproliferative mutant cell line, showed none of the same signs of damage as in the iKRas cells beyond minor elevation in p21^{waf1/cip1} expression.

Unbiased biochemical profiling allowed the inventors to identify a unique bioactive enzyme isoform of the broad secreted Pla2 gene family, PLA2G7, as a mediator of the damage. Confocal imaging showed substantial overexpression of group 7A sPLA2, while RNAseq reported a 32-fold elevation of Pla2g7 gene expression but not that of Pafah2 (group 7B), its homolog. Using a nanosensor which detects lipids in the endosomal lumen of living cells, the inventors explored lipid dysregulation in this system. Using a fluorescent nanosensor to benchmark lipid accumulation, the inventors found that oncogenic RAS amplification induces substantial lipid dysregulation in the intracellular endosomal compartments as well as endomembrane trafficking defects. Only specific inhibitors of group 7 sPLA2 and RAS-ERK signaling abrogated the lipid dysregulation. Using the nanosensor, the inventors found partial intracellular lipid dysregulation in eKRas, the endogenous mutant; however, eKRas showed no significant stress response or group 7A expression. These findings resemble the dosage-dependent nature of this oncogene, but also importantly—because constitutive RAS signaling can directly generate oxidants^{14, 18, 58, 59}—these findings support that the partial lipid phenotype and mild upregulation of p21^{waf1/cip1} in eKRas reflects oxidized phospholipid substrate generation that was insufficient to trigger group 7A. A link between p21^{waf1/cip1} expression, cellular senescence, and oxidative damage feedback has been shown.^{60, 61} Stress and inflammation inducible group 7A isoforms may require the highly-elevated stress of RAS amplification for expression, even though these isoforms are paradoxically inactivated by direct oxidative attack.⁶²

The inventors further investigated the source and identity of the lipids involved in the aberrant accumulation. It was found that culture medium-derived polyunsaturated fatty acids (PUFA) were necessary for intracellular lipid dysregulation, which is expected, as membrane-active enzymes in the Pla2 family play an important role in PUFA release^{46, 63} while group 7 enzymes selectively attack membrane lipids with oxidized PUFA.^{64, 65, 66, 67} The sensitivity of intracellular lipid dysregulation on PUFA loading and lipophilic anti-oxidant treatment is consistent with the membrane damage responsive role of group 7 enzymes.

The main products of group 7 sPLA2 enzyme catalysis are soluble lysophospholipids and oxidized fatty acids; this catalytic activity should stop when the enzymes are degraded. However, the inventors attempts to block endolysosomal degradation had no influence on lipid dysregulation (FIG. 23). Without being bound by theory, this can be explained by the fact that group 7 enzymes are acid-labile and inactivated at acidic pH^{68, 69} while endomembrane compartments in Ras-transformed fibroblasts are alkaline⁷⁰ and support enzyme activity.

A link between inflammatory group 7A activity and RAS-mediated survival was tested with the use of a non-degradable PAF-analog substrate lipid. The inventors evidenced a link between an important marker of oncogene-induced growth delay, p21^{waf1/cip1}, and intracellular accumulation of this cytostatic substrate lipid class. Based on a model where cytotoxic substrate lipid must be cleared out of the membrane for survival, the data discussed in this disclosure indeed supported this since group 7 gene silencing did not abrogate p21^{waf1/cip1} expression, while chemical inhibition of available group 7 enzymes promoted p21^{waf1/cip1} expression. Importantly, the inventors showed group 7 gene silencing selectively killed RAS-damaged, but not control, cells.

Fibroblasts, including embryonic lines, are an often-used model system to study ras-transformation and, more recently, SIR/SASP phenomena induced by senescence-associated stresses. Fibroblasts can synthesize canonical PAF under specific conditions or generate IL-6 in the presence of critical micelle concentrations of exogenous PAF lipid:^{71, 72} Here, the inventors found RAS overexpression led to elevated IL-6 levels in cell lysates, supporting that damage through oncogenic RAS amplification stimulates conserved inflammatory responses. It is interesting to note that damage-associated molecular patterns in leukocytes' and DNA damage signaling from ultraviolet radiation^{74, 75} also impinge on a group 7/PAF/p21^{waf1/cip1} pathway in which oxidative modifications are key traits. While the inventors did not interrogate the downstream details of group 7 activation or transcription in this cellular context, recent evidence from the study of NRF2 activation and anti-oxidant gene expression during RAS carcinogenesis suggests that combating oxidative stress and damage, including maintaining low levels of oxidation, are critical events for the survival and proliferation of endogenous RAS mutant cells.⁷⁶ Severe damage is more likely to halt cell growth; therefore, mechanisms that repair this damage would select for aggressive cells.

While PLA2G7A is a soluble lipoprotein-associated serine hydrolase derived largely from leukocytes, PLA2G7B (pafah2) transfers from cytosol to ER membranes and was first described as an oxidant detoxifier in liver,^{65, 77} erythrocytes,⁷⁸ and the yeast S. pombe.⁶⁴ Both enzymes are thought to act at the aqueous side of the membrane-water interface where most substrates are located, although it is possible activity against cytosolic substrates is relevant. At least part of this activity is endomembrane localized to endosomal compartments.

Based on the nanosensor and lipidomics findings that the appearance of soluble lysophospholipids are linked to group 7 activity, the inventors investigated inhibitors of group 7 sPLA2 enzymes. It was found that group 7 inhibitors can selectively kill RAS-overexpressing cells. The most developed compound for these stress enzymes is darapladib, a group 7A and 7B inhibitor, which reached Phase 3 trials before failing to meet its intended cardiovascular clinical endpoint. While darapladib is safe and apparently side-effect-free in vivo, most cells harbor the group 7B enzyme for its housekeeper enzymatic functions. Therefore, blanket inhibition could selectively affect cell types where membrane damage is significant, or turnover is slow. Many human neoplasms overexpress or amplify RAS, including the point mutation leading to constitutive signaling.

### REFERENCES

1. Bihani T, Chicas A, Lo C P, Lin A W. Dissecting the senescence-like program in tumor cells activated by Ras signaling. The Journal of biological chemistry 282, 2666-2675 (2007).
2. Zhu J, Woods D, McMahon M, Bishop J M. Senescence of human fibroblasts induced by oncogenic Raf. Genes Dev 12, 2997-3007 (1998).
3. Dimauro T, David G. Ras-induced senescence and its physiological relevance in cancer. Current cancer drug targets 10, 869-876 (2010).
4. Serrano M, Lin A W, McCurrach M E, Beach D, Lowe S W. Oncogenic ras Provokes Premature Cell Senescence Associated with Accumulation of p53 and p16INK4a. Cell 88, 593-602 (1997).
5. Lin A W, Barradas M, Stone J C, van Aelst L, Serrano M, Lowe S W. Premature senescence involving p53 and p16 is activated in response to constitutive MEK/MAPK mitogenic signaling. Genes & Development 12, 3008-3019 (1998).
6. Sewing A, Wiseman B, Lloyd A C, Land H. High-intensity Raf signal causes cell cycle arrest mediated by p21Cip1. Molecular and cellular biology 17, 5588-5597 (1997).
7. Deschenes-Simard X, et al. Tumor suppressor activity of the ERK/MAPK pathway by promoting selective protein degradation. Genes Dev 27, 900-915 (2013).
8. Shin J, Yang J, Lee J C, Baek K H. Depletion of ERK2 but not ERK1 abrogates oncogenic Ras-induced senescence. Cell Signal 25, 2540-2547 (2013).
9. Bartkova J, et al. Oncogene-induced senescence is part of the tumorigenesis barrier imposed by DNA damage checkpoints. Nature 444, 633-637 (2006).
10. Collado M, et al. Tumour biology: senescence in premalignant tumours. Nature 436, 642 (2005).
11. Braig M, et al. Oncogene-induced senescence as an initial barrier in lymphoma development. Nature 436, 660-665 (2005).
12. Michaloglou C, et al. BRAFE600-associated senescence-like cell cycle arrest of human naevi. Nature 436, 720-724 (2005).
13. Sarkisian C J, Keister B A, Stairs D B, Boxer R B, Moody S E, Chodosh L A. Dose-dependent oncogene-induced senescence in vivo and its evasion during mammary tumorigenesis. Nature cell biology 9, 493-505 (2007).
14. Lee A C, et al. Ras proteins induce senescence by altering the intracellular levels of reactive oxygen species. The Journal of biological chemistry 274, 7936-7940 (1999).
15. Ogrunc M, et al. Oncogene-induced reactive oxygen species fuel hyperproliferation and DNA damage response activation. Cell Death And Differentiation 21, 998 (2014).
16. Di Micco R, et al. Oncogene-induced senescence is a DNA damage response triggered by DNA hyper-replication. Nature 444, 638-642 (2006).
17. Blagosklonny M V. Cell senescence and hypermitogenic arrest. EMBO reports 4, 358-362 (2003).
18. Weyemi U, et al. ROS-generating NADPH oxidase NOX4 is a critical mediator in oncogenic H-Ras-induced DNA damage and subsequent senescence. Oncogene 31, 1117-1129 (2012).
19. Tuveson D A, et al. Endogenous oncogenic K-ras(G12D) stimulates proliferation and widespread neoplastic and developmental defects. Cancer Cell 5, 375-387 (2004).
20. Chen X, et al. Endogenous expression of Hras(G12V) induces developmental defects and neoplasms with copy number imbalances of the oncogene. Proceedings of the National Academy of Sciences of the United States of America 106, 7979-7984 (2009).
21. Guerra C, et al. Tumor induction by an endogenous K-ras oncogene is highly dependent on cellular context. Cancer Cell 4, 111-120 (2003).
22. Schuhmacher A J, Guerra C, Sauzeau V, Cañamero M, Bustelo X R, Barbacid M. A mouse model for Costello syndrome reveals an Ang II-mediated hypertensive condition. The Journal of Clinical Investigation 118, 2169-2179 (2008).
23. Coppé J-P, et al. Senescence-Associated Secretory Phenotypes Reveal Cell-Nonautonomous Functions of Oncogenic RAS and the p53 Tumor Suppressor. PLOS Biology 6, e301 (2008).
24. Freund A, Orjalo A V, Desprez P Y, Campisi J. Inflammatory networks during cellular senescence: causes and consequences. Trends Mol Med 16, 238-246 (2010).
25. Lee S J, et al. Senescing Human Bone-Marrow-Derived Clonal Mesenchymal Stem Cells Have Altered Lysophospholipid Composition and Functionality. Journal of Proteome Research 13, 1438-1449 (2014).
26. Augert A, Payré C, de Launoit Y, Gil J, Lambeau G, Bernard D. The M-type receptor PLA2R regulates senescence through the p53 pathway. EMBO reports 10, 271-277 (2009).
27. Kim H J, et al. Induction of cellular senescence by secretory phospholipase A2 in human dermal fibroblasts through an ROS-mediated p53 pathway. The journals of gerontology Series A, Biological sciences and medical sciences 64, 351-362 (2009).
28. Pribluda A, et al. A senescence-inflammatory switch from cancer-inhibitory to cancer-promoting mechanism. Cancer Cell 24, 242-256 (2013).
29. Kim S S, Kim D K, Suh Y H. Cerebral cortical phospholipase A2 activity of senescence-accelerated mouse is increased in an age-dependent manner. Neuroscience research 29, 269-272 (1997).
30. Murakami M, Sato H, Miki Y, Yamamoto K, Taketomi Y. A new era of secreted phospholipase A(2). J Lipid Res 56, 1248-1261 (2015).
31. Scott K F, et al. Emerging roles for phospholipase A2 enzymes in cancer. Biochimie 92, 601-610 (2010).
32. Murakami M, Taketomi Y, Girard C, Yamamoto K, Lambeau G. Emerging roles of secreted phospholipase A2 enzymes: Lessons from transgenic and knockout mice. Biochimie 92, 561-582 (2010).
33. Lasry A, Ben-Neriah Y. Senescence-associated inflammatory responses: aging and cancer perspectives. Trends in Immunology 36, 217-228 (2015).
34. Martien S, et al. Cellular senescence involves an intracrine prostaglandin E2 pathway in human fibroblasts. Biochimica et Biophysica Acta (BBA)—Molecular and Cell Biology of Lipids 1831, 1217-1227 (2013).
35. Freund A, Patil C K, Campisi J. p38MAPK is a novel DNA damage response-independent regulator of the senescence-associated secretory phenotype. The EMBO journal 30, 1536-1548 (2011).
36. Coppé J-P, Desprez P-Y, Krtolica A, Campisi J. The senescence-associated secretory phenotype: the dark side of tumor suppression. Annu Rev Pathol 5, 99-118 (2010).
37. Kuilman T, et al. Oncogene-induced senescence relayed by an interleukin-dependent inflammatory network. Cell 133, 1019-1031 (2008).
38. Thevenin A F, Monillas E S, Winget J M, Czymmek K, Bahnson B J. Trafficking of platelet-activating factor acetylhydrolase type II in response to oxidative stress. Biochemistry 50, 8417-8426 (2011).
39. Galassi T V, et al. An optical nanoreporter of endolysosomal lipid accumulation reveals enduring effects of diet on hepatic macrophages in vivo. Science Translational Medicine 10, eaar2680 (2018).
40. Jena P V, et al. A Carbon Nanotube Optical Reporter Maps Endolysosomal Lipid Flux. ACS nano, (2017).
41. Joseph M G Nagano K-L H, Anna E Speers, Steven J Brown, Timothy Spicer, Virneliz Fernandez-Vega, Jill Ferguson, Brian J Bahnson, Benjamin F Cravatt, Peter Hodder, and Hugh Rosen. Optimization and characterization of a carbamate inhibitor for plasma platelet-activating factor acetylhydrolase (pPAFAH). Probe Reports from the NIH Molecular Libraries Program, (2011).
42. Alexander Adibekian K-L H, Anna E Speers, Elizabeth S Monillas, Steven J Brown, Timothy Spicer, Virneliz Fernandez-Vega, Jill Ferguson, Brian J Bahnson, Benjamin F Cravatt, Peter Hodder, and Hugh Rosen. Optimization and characterization of a triazole urea inhibitor for platelet-activating factor acetylhydrolase type 2 (PAFAH2). Probe Reports from the NIH Molecular Libraries Program, (2011).
43. Fisher A B, Dodia C, Chander A, Jain M. A competitive inhibitor of phospholipase A&lt; sub&gt; 2&lt;/sub&gt; decreases surfactant phosphatidylcholine degradation by the rat lung. Biochemical Journal 288, 407 (1992).
44. Prescott S M, Zimmerman G A, Stafforini D M, McIntyre T M. Platelet-Activating Factor and Related Lipid Mediators. Annual Review of Biochemistry 69, 419-445 (2000).
45. Howard A C, McNeil A K, McNeil P L. Promotion of plasma membrane repair by vitamin E. Nature Communications 2, 597 (2011).
46. Snyder F. Platelet-activating factor: the biosynthetic and catabolic enzymes. The Biochemical journal 305 (Pt 3), 689-705 (1995).
47. Schmitz G, Müller G. Structure and function of lamellar bodies, lipid-protein complexes involved in storage and secretion of cellular lipids. Journal of lipid research 32, 1539-1570 (1991).
48. Kashiwagi M, et al. Group II and IV phospholipase A2 are produced in human pancreatic cancer cells and influence prognosis. Gut 45, 605-612 (1999).
49. Cormier R T, et al. Secretory phospholipase Pla2g2a confers resistance to intestinal tumorigenesis. Nature genetics 17, 88-91 (1997).
50. Fijneman R J, et al. Pla2g2a attenuates colon tumorigenesis in azoxymethane-treated C57BL/6 mice; expression studies reveal Pla2g2a target genes and pathways. Cellular oncology: the official journal of the International Society for Cellular Oncology 31, 345-356 (2009).
51. Hiyoshi M, et al. The expression of phospholipase A2 group X is inversely associated with metastasis in colorectal cancer. Oncology Letters 5, 533-538 (2013).
52. Sadaria M R, Yu J A, Meng X, Fullerton D A, Reece T B, Weyant M J. Secretory Phospholipase A2 Mediates Human Esophageal Adenocarcinoma Cell Growth and Proliferation via ERK 1/2 Pathway. Anticancer Research 33, 1337-1342 (2013).
53. Yu J A, et al. Group IIa secretory phospholipase expression correlates with group IIa secretory phospholipase inhibition-mediated cell death in K-ras mutant lung cancer cells. The Journal of thoracic and cardiovascular surgery 144, 1479-1485 (2012).
54. Dimitrova N, et al. Stromal Expression of miR-143/145 Promotes Neoangiogenesis in Lung Cancer Development. Cancer Discov 6, 188-201 (2016).
55. Ruscetti M, et al. N K cell-mediated cytotoxicity contributes to tumor control by a cytostatic drug combination. Science (New York, N.Y.) 362, 1416-1422 (2018).
56. Acosta J C, et al. A complex secretory program orchestrated by the inflammasome controls paracrine senescence. Nature cell biology 15, 978-990 (2013).
57. Lasry A, Aran D, Butte A J, Ben-Neriah Y. Cancer Cell—Autonomous Parainflammation Mimics Immune Cell Infiltration. Cancer Research 77, 3740-3744 (2017).
58. Kong B, Qia C, Erkan M, Kleeff J, Michalski C W. Overview on how oncogenic Kras promotes pancreatic carcinogenesis by inducing low intracellular ROS levels. Frontiers in Physiology 4, (2013).
59. Irani K, et al. Mitogenic signaling mediated by oxidants in Ras-transformed fibroblasts. Science (New York, N.Y.) 275, 1649-1652 (1997).
60. Macip S, et al. Inhibition of p21-mediated ROS accumulation can rescue p21-induced senescence. The EMBO journal 21, 2180-2188 (2002).
61. Passos J F, et al. Feedback between p21 and reactive oxygen production is necessary for cell senescence. Molecular Systems Biology 6, 347 (2010).
62. Ambrosio G, et al. Oxygen radicals inhibit human plasma acetylhydrolase, the enzyme that catabolizes platelet-activating factor. J Clin Invest 93, 2408-2416 (1994).
63. Leslie C C, Gelb M H. Assaying Phospholipase A2 Activity. In: Signal Transduction Protocols (eds Dickson R C, Mendenhall M D). Humana Press (2004).
64. Foulks J M, Weyrich A S, Zimmerman G A, McIntyre T M. A yeast PAF acetylhydrolase ortholog suppresses oxidative death. Free radical biology & medicine 45, 434-442 (2008).
65. Kono N, et al. Protection against oxidative stress-induced hepatic injury by intracellular type II platelet-activating factor acetylhydrolase by metabolism of oxidized phospholipids in vivo. The Journal of biological chemistry 283, 1628-1636 (2008).
66. MacPhee C H, et al. Lipoprotein-associated phospholipase A2, platelet-activating factor acetylhydrolase, generates two bioactive products during the oxidation of low-density lipoprotein: use of a novel inhibitor. The Biochemical journal 338 (Pt 2), 479-487 (1999).
67. Matsuzawa A, Hattori K, Aoki J, Arai H, Inoue K. Protection against oxidative stress-induced cell death by intracellular platelet-activating factor-acetylhydrolase II. The Journal of biological chemistry 272, 32315-32320 (1997).
68. Farr R S, Wardlow M L, Cox C P, Meng K E, Greene D E. Human serum acid-labile factor is an acylhydrolase that inactivates platelet-activating factor. Federation proceedings 42, 3120-3122 (1983).
69. Farr R S, Cox C P, Wardlow M L, Jorgensen R. Preliminary studies of an acid-labile factor (ALF) in human sera that inactivates platelet-activating factor (PAF). Clinical Immunology and Immunopathology 15, 318-330 (1980).
70. Jiang L W, Maher V M, McCormick J J, Schindler M. Alkalinization of the lysosomes is correlated with ras transformation of murine and human fibroblasts. Journal of Biological Chemistry 265, 4775-4777 (1990).
71. Michel L, et al. Biosynthesis of paf-acether factor-acether by human skin fibroblasts in vitro. The Journal of Immunology 141, 948 (1988).
72. Braquet P, Pignol B, Maisonnet T, Mencia-Huerta J M. Platelet-activating factor modulates interleukin-6 production by mouse fibroblasts. International archives of allergy and applied immunology 94, 165-166 (1991).
73. Cao Y, Stafforini D M, Zimmerman G A, McIntyre T M, Prescott S M. Expression of plasma platelet-activating factor acetylhydrolase is transcriptionally regulated by mediators of inflammation. The Journal of biological chemistry 273, 4012-4020 (1998).
74. Damiani E, Puebla-Osorio N, Lege B M, Liu J, Neelapu S S, Ullrich S E. Platelet activating factor-induced expression of p21 is correlated with histone acetylation. Scientific Reports 7, 41959 (2017).
75. Walterscheid J P, Ullrich S E, Nghiem D X. Platelet-activating Factor, a Molecular Sensor for Cellular Damage, Activates Systemic Immune Suppression. The Journal of Experimental Medicine 195, 171-179 (2002).
76. Lim J K M, Leprivier G. The impact of oncogenic RAS on redox balance and implications for cancer development. Cell Death & Disease 10, 955 (2019).
77. Hattori K, et al. cDNA Cloning and Expression of Intracellular Platelet-activating Factor (PAF) Acetylhydrolase II: ITS HOMOLOGY WITH PLASMA PAF ACETYLHYDROLASE. Journal of Biological Chemistry 271, 33032-33038 (1996).
78. Stafforini D M, Rollins E N, Prescott S M, McIntyre T M. The platelet-activating factor acetylhydrolase from human erythrocytes. Purification and properties. The Journal of biological chemistry 268, 3857-3865 (1993).
79. Smith M W, et al. Identification of Novel Tumor Markers in Hepatitis C Virus-associated Hepatocellular Carcinoma. Cancer Research 63, 859 (2003).
80. Xu C, et al. Deficiency of phospholipase A2 group 7 decreases intestinal polyposis and colon tumorigenesis in Apc(Min/+) mice. Cancer Res 73, 2806-2816 (2013).
81. Denizot Y, Gainant A, Guglielmi L, Bouvier S, Cubertafond P, Mathonnet M. Tissue concentrations of platelet-activating factor in colorectal carcinoma: inverse relationships with Dukes' stage of patients. Oncogene 22, 7222-7224 (2003).
82. Cheong H, Lindsten T, Wu J, Lu C, Thompson C B. Ammonia-induced autophagy is independent of ULK1/ULK2 kinases. Proceedings of the National Academy of Sciences of the United States of America 108, 11121-11126 (2011).
83. Wei M C, et al. Proapoptotic BAX and BAK: a requisite gateway to mitochondrial dysfunction and death. Science (New York, N.Y.) 292, 727-730 (2001).
84. Palm W, Park Y, Wright K, Pavlova N N, Tuveson D A, Thompson C B. The Utilization of Extracellular Proteins as Nutrients Is Suppressed by mTORC1. Cell 162, 259-270 (2015).
85. Zuber J, et al. Toolkit for evaluating genes required for proliferation and survival using tetracycline-regulated RNAi. Nature biotechnology 29, 79-83 (2011).
86. Palm W, Araki J, King B, DeMatteo R G, Thompson C B. Critical role for PI3-kinase in regulating the use of proteins as an amino acid source. Proceedings of the National Academy of Sciences 114, E8628-E8636 (2017).
87. Roxbury D, et al. Hyperspectral Microscopy of Near-Infrared Fluorescence Enables 17-Chirality Carbon Nanotube Imaging. Scientific Reports 5, (2015).
88. Jena P V, et al. A Carbon Nanotube Optical Reporter Maps Endolysosomal Lipid Flux. ACS nano 11, 10689-10703 (2017).
89. Roxbury D, Jena P V, Shamay Y, Horoszko C P, Heller D A. Cell membrane proteins modulate the carbon nanotube optical bandgap via surface charge accumulation. ACS Nano 10, 499-506 (2016).
90. Ruscetti M, et al. N K cell-mediated cytotoxicity contributes to tumor control by a cytostatic drug combination. Science 362, 1416 (2018).
91. Gao J, et al. Integrative Analysis of Complex Cancer Genomics and Clinical Profiles Using the cBioPortal. Science Signaling 6, pl 1 (2013).
92. Cerami E, et al. The cBio Cancer Genomics Portal: An Open Platform for Exploring Multidimensional Cancer Genomics Data. Cancer Discovery 2, 401 (2012).

While certain embodiments have been illustrated and described, a person with ordinary skill in the art, after reading the foregoing specification, can effect changes, substitutions of equivalents and other types of alterations to the compounds of the present technology or salts, pharmaceutical compositions, derivatives, prodrugs, metabolites, tautomers, or racemic mixtures thereof as set forth herein. Each aspect and embodiment described above can also have included or incorporated therewith such variations or aspects as disclosed in regard to any or all of the other aspects and embodiments.

The present technology is also not to be limited in terms of the particular aspects described herein, which are intended as single illustrations of individual aspects of the present technology. Many modifications and variations of this present technology can be made without departing from its spirit and scope, as will be apparent to those skilled in the art. Functionally equivalent methods within the scope of the present technology, in addition to those enumerated herein, will be apparent to those skilled in the art from the foregoing descriptions. Such modifications and variations are intended to fall within the scope of the appended claims. It is to be understood that this present technology is not limited to particular methods, reagents, compounds, compositions, labeled compounds or biological systems, which can, of course, vary. It is also to be understood that the terminology used herein is for the purpose of describing particular aspects only, and is not intended to be limiting. Thus, it is intended that the specification be considered as exemplary only with the breadth, scope and spirit of the present technology indicated only by the appended claims, definitions therein and any equivalents thereof.

The embodiments, illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the claimed technology. Additionally, the phrase “consisting essentially of” will be understood to include those elements specifically recited and those additional elements that do not materially affect the basic and novel characteristics of the claimed technology. The phrase “consisting of” excludes any element not specified.

In addition, where features or aspects of the disclosure are described in terms of Markush groups, those skilled in the art will recognize that the disclosure is also thereby described in terms of any individual member or subgroup of members of the Markush group. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member.

All publications, patent applications, issued patents, and other documents (for example, journals, articles and/or textbooks) referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

The present technology may include, but is not limited to, the features and combinations of features recited in the following lettered paragraphs, it being understood that the following paragraphs should not be interpreted as limiting the scope of the claims as appended hereto or mandating that all such features must necessarily be included in such claims:
A. A method of treating a cancer in a subject, the method comprising administering to the subject an effective amount of a compound to treat the cancer; wherein the compound is at least one of darapladib, rilapladib, AA39-2, or ML256; and wherein the cancer harbors a constitutively active variant of one or both of KRAS or HRAS, wherein the constitutively active variant is a gain of function mutation, a duplication, or a gene amplification; optionally wherein the cancer exhibits elevated expression and/or activity of at least one of PLA2G7A and PAFAH2 compared to healthy control cells.
B. The method of Paragraph A, wherein the cancer is a pancreatic cancer, a colorectal cancer, a hepatocellular cancer, a bile duct cancer, a soft tissue sarcoma, a blood or hematopoietic cell cancer, a breast cancer, a lung cancer, a uterine or cervical cancer, a thyroid cancer, a bladder cancer, a kidney cancer, a gastric cancer, an ovarian cancer, a brain cancer, a mesothelioma cancer, a skin cancer, a head and neck cancer, a neuroendocrine cancer or neoplasm, an esophagus cancer, a testicular cancer, a prostate cancer, or a thymus cancer.
C. The method of Paragraph A or Paragraph B, wherein the cancer comprises an adenoma, an adenocarcinoma, a uterine carcinoma, a squamous cell carcinoma, a small cell carcinoma, a transitional carcinoma, a serous carcinoma, a clear-cell carcinoma, a mucinous adenocarcinoma, an undifferentiated carcinoma, a dedifferentiated carcinoma, a serous adenocarcinoma, a sarcoma, a myeloma, a leukemia, a lymphoma, a dysplastic lesion, or a combination of any two or more thereof.
D. The method of any one of Paragraphs A-C, wherein the subject is human.
E. The method of any one of Paragraphs A-D, wherein the method comprises administering to the subject (in terms of mass of the compound/mass of the subject) about 0.01 mg/kg to about 20 mg/kg of the compound.
F. The method of any one of Paragraphs A-E, wherein the method comprises administering to the subject (in terms of mass of the compound/mass of the subject) about 0.25 mg/kg to about 10 mg/kg of the compound.
G. The method of any one of Paragraphs A-F, wherein the compound is darapladib.
H. The method of any one of Paragraphs A-F, wherein the compound is rilapladib.
I. The method of any one of Paragraphs A-F, wherein the compound is AA39-2.
J. The method of any one of Paragraphs A-F, wherein the compound is ML256.
K. The method of any one of Paragraphs A-J, wherein the administering further comprises administration of a chemotherapeutic agent selected from the group consisting of an alkylating agent; a nitrosourea; an antimetabolite; an anthracycline; a topoisomerase II inhibitor; a mitotic inhibitor; an anti-estrogen; a progestin; an aromatase inhibitor; an anti-androgen; an LHRH agonist; a corticosteroid hormone; a DNA alkylating agent; a taxane; a vinca alkaloid; a microtubule poison, and a combination of any two or more thereof.
L. The method of any one of Paragraphs A-K, wherein the administering further comprises administration of a chemotherapeutic agent selected from the group consisting of busulfan, cisplatin, carboplatin, oxaliplatin, an octahedral platinum (IV) compound, chlorambucil, cyclophosphamide, ifosfamide, dacarbazine (DTIC), mechlorethamine (nitrogen mustard), melphalan, temozolomide, carmustine (BCNU), lomustine (CCNU), 5-fluorouracil, capecitabine, 6-mercaptopurine, methotrexate, gemcitabine, cytarabine (ara-C), fludarabine, pemetrexed, daunorubicin, doxorubicin (Adriamycin), epirubicin, idarubicin, mitoxantrone, topotecan, irinotecan, etoposide (VP-16), teniposide, paclitaxel, docetaxel, vinblastine, vincristine, vinorelbine, prednisone, dexamethasone, L-asparaginase, dactinomycin, thalidomide, tretinoin, imatinib (Gleevec), gefitinib (Iressa), erlotinib (Tarceva), rituximab (Rituxan), bevacizumab (Avastin), ipilimumab, nivolumab (Opdivo), pembrolizumab (Ketruda), tamoxifen, fulvestrant, anastrozole, exemestane, letrozole, megestrol acetate, bicalutamide, flutamide, leuprolide, goserelin, and a combination of any two or more thereof.
M. The method of any one of Paragraphs A-L, wherein the administering comprises oral administration, intravenous administration, or intramuscular administration.
N. The method of any one of Paragraphs A-M, wherein the method comprises orally administering to the subject the effective amount of the compound to treat the cancer.
O. A method of slowing or reversing growth of a tumor in a subject, the method comprising administering to the subject an effective amount of a compound; wherein the compound is at least one of darapladib, rilapladib, AA39-2, or ML256; wherein the effective amount is an amount effective to slow or reverse growth of the tumor; and wherein the tumor is of a cancer that harbors a constitutively active variant of one or both of KRAS or HRAS, wherein the constitutively active variant is a gain of function mutation, a duplication, or a gene amplification; optionally wherein the cancer exhibits elevated expression and/or activity of at least one of PLA2G7A and PAFAH2 compared to healthy control cells.
P. The method of Paragraph O, wherein the tumor is of a cancer selected from a pancreatic cancer, a colorectal cancer, a hepatocellular cancer, a bile duct cancer, a soft tissue sarcoma, a blood or hematopoietic cell cancer, a breast cancer, a lung cancer, a uterine or cervical cancer, a thyroid cancer, a bladder cancer, a kidney cancer, a gastric cancer, an ovarian cancer, a brain cancer, a mesothelioma cancer, a skin cancer, a head and neck cancer, a neuroendocrine cancer or neoplasm, an esophagus cancer, a testicular cancer, a prostate cancer, or a thymus cancer.
Q. The method of Paragraph O or Paragraph P, wherein the tumor is of a cancer comprising an adenoma, an adenocarcinoma, a uterine carcinoma, a squamous cell carcinoma, a small cell carcinoma, a transitional carcinoma, a serous carcinoma, a clear-cell carcinoma, a mucinous adenocarcinoma, an undifferentiated carcinoma, a dedifferentiated carcinoma, a serous adenocarcinoma, a sarcoma, a myeloma, a leukemia, a lymphoma, a dysplastic lesion, or a combination of any two or more thereof.
R. The method of any one of Paragraphs O-Q, wherein the subject is human.
S. The method of any one of Paragraphs O-R, wherein the method comprises administering to the subject (in terms of mass of the compound/mass of the subject) about 0.01 mg/kg to about 20 mg/kg of the compound.
T. The method of any one of Paragraphs O-S, wherein the method comprises administering to the subject (in terms of mass of the compound/mass of the subject) about 0.25 mg/kg to about 10 mg/kg of the compound.
U. The method of any one of Paragraphs O-T, wherein the compound is darapladib.
V. The method of any one of Paragraphs O-T, wherein the compound is rilapladib.
W. The method of any one of Paragraphs O-T, wherein the compound is AA39-2.
X. The method of any one of Paragraphs O-T, wherein the compound is ML256.
Y. The method of any one of Paragraphs O-X, wherein the administering further comprises administration of a chemotherapeutic agent selected from the group consisting of an alkylating agent; a nitrosourea; an antimetabolite; an anthracycline; a topoisomerase II inhibitor; a mitotic inhibitor; an anti-estrogen; a progestin; an aromatase inhibitor; an anti-androgen; an LHRH agonist; a corticosteroid hormone; a DNA alkylating agent; a taxane; a vinca alkaloid; a microtubule poison, and a combination of any two or more thereof.
Z. The method of any one of Paragraphs O-Y, wherein the administering further comprises administration of a chemotherapeutic agent selected from the group consisting of busulfan, cisplatin, carboplatin, oxaliplatin, an octahedral platinum (IV) compound, chlorambucil, cyclophosphamide, ifosfamide, dacarbazine (DTIC), mechlorethamine (nitrogen mustard), melphalan, temozolomide, carmustine (BCNU), lomustine (CCNU), 5-fluorouracil, capecitabine, 6-mercaptopurine, methotrexate, gemcitabine, cytarabine (ara-C), fludarabine, pemetrexed, daunorubicin, doxorubicin (Adriamycin), epirubicin, idarubicin, mitoxantrone, topotecan, irinotecan, etoposide (VP-16), teniposide, paclitaxel, docetaxel, vinblastine, vincristine, vinorelbine, prednisone, dexamethasone, L-asparaginase, dactinomycin, thalidomide, tretinoin, imatinib (Gleevec), gefitinib (Iressa), erlotinib (Tarceva), rituximab (Rituxan), bevacizumab (Avastin), ipilimumab, nivolumab (Opdivo), pembrolizumab (Ketruda), tamoxifen, fulvestrant, anastrozole, exemestane, letrozole, megestrol acetate, bicalutamide, flutamide, leuprolide, goserelin, and a combination of any two or more thereof.
AA. The method of any one of Paragraphs O-Z, wherein the administering comprises oral administration, intravenous administration, or intramuscular administration.
AB. The method of any one of Paragraphs O-AA, wherein the method comprises orally administering to the subject the effective amount of the compound.
AC. The method of any one of Paragraphs O-AB, wherein the administering comprises injection of the compound into the tumor or proximal to the tumor.
AD. A method of slowing or reversing growth of a neoplasm in a subject and/or slowing or reversing proliferation of the neoplasm in the subject, the method comprising administering to the subject an effective amount of a compound; where the compound is at least one of darapladib, rilapladib, AA39-2, or ML256; wherein the effective amount is an amount effective to slow or reverse growth of the neoplasm and/or slow or reverse proliferation of the neoplasm; and wherein the neoplasm is of a cancer that harbors a constitutively active variant of one or both of KRAS or HRAS, wherein the constitutively active variant is a gain of function mutation, a duplication, or a gene amplification; optionally wherein the cancer exhibits elevated expression and/or activity of at least one of PLA2G7A and PAFAH2 compared to healthy control cells.
AE. The method of Paragraph AD, wherein the neoplasm is of a cancer selected from a pancreatic cancer, a colorectal cancer, a hepatocellular cancer, a bile duct cancer, a soft tissue sarcoma, a blood or hematopoietic cell cancer, a breast cancer, a lung cancer, a uterine or cervical cancer, a thyroid cancer, a bladder cancer, a kidney cancer, a gastric cancer, an ovarian cancer, a brain cancer, a mesothelioma cancer, a skin cancer, a head and neck cancer, a neuroendocrine cancer or neoplasm, an esophagus cancer, a testicular cancer, a prostate cancer, or a thymus cancer.
AF. The method of Paragraph AD or Paragraph AE, wherein the neoplasm is of a cancer comprising an adenoma, an adenocarcinoma, a uterine carcinoma, a squamous cell carcinoma, a small cell carcinoma, a transitional carcinoma, a serous carcinoma, a clear-cell carcinoma, a mucinous adenocarcinoma, an undifferentiated carcinoma, a dedifferentiated carcinoma, a serous adenocarcinoma, a sarcoma, a myeloma, a leukemia, a lymphoma, a dysplastic lesion, or a combination of any two or more thereof.
AG. The method of any one of Paragraphs AD-AF, wherein the subject is human.
AG. The method of any one of Paragraphs AD-AG, wherein the method comprises administering to the subject (in terms of mass of the compound/mass of the subject) about 0.01 mg/kg to about 20 mg/kg of the compound.
AI. The method of any one of Paragraphs AD-AH, wherein the method comprises administering to the subject (in terms of mass of the compound/mass of the subject) about 0.25 mg/kg to about 10 mg/kg of the compound.
AJ. The method of any one of Paragraphs AD-AI, wherein the compound is darapladib.
AK. The method of any one of Paragraphs AD-AI, wherein the compound is rilapladib.
AL. The method of any one of Paragraphs AD-AI, wherein the compound is AA39-2.
AM. The method of any one of Paragraphs AD-AI, wherein the compound is ML256.
AN. The method of any one of Paragraphs AD-AM, wherein the administering further comprises administration of a chemotherapeutic agent selected from the group consisting of an alkylating agent; a nitrosourea; an antimetabolite; an anthracycline; a topoisomerase II inhibitor; a mitotic inhibitor; an anti-estrogen; a progestin; an aromatase inhibitor; an anti-androgen; an LHRH agonist; a corticosteroid hormone; a DNA alkylating agent; a taxane; a vinca alkaloid; a microtubule poison, and a combination of any two or more thereof.
AO. The method of any one of Paragraphs AD-AN, wherein the administering further comprises administration of a chemotherapeutic agent selected from the group consisting of busulfan, cisplatin, carboplatin, oxaliplatin, an octahedral platinum (IV) compound, chlorambucil, cyclophosphamide, ifosfamide, dacarbazine (DTIC), mechlorethamine (nitrogen mustard), melphalan, temozolomide, carmustine (BCNU), lomustine (CCNU), 5-fluorouracil, capecitabine, 6-mercaptopurine, methotrexate, gemcitabine, cytarabine (ara-C), fludarabine, pemetrexed, daunorubicin, doxorubicin (Adriamycin), epirubicin, idarubicin, mitoxantrone, topotecan, irinotecan, etoposide (VP-16), teniposide, paclitaxel, docetaxel, vinblastine, vincristine, vinorelbine, prednisone, dexamethasone, L-asparaginase, dactinomycin, thalidomide, tretinoin, imatinib (Gleevec), gefitinib (Iressa), erlotinib (Tarceva), rituximab (Rituxan), bevacizumab (Avastin), ipilimumab, nivolumab (Opdivo), pembrolizumab (Ketruda), tamoxifen, fulvestrant, anastrozole, exemestane, letrozole, megestrol acetate, bicalutamide, flutamide, leuprolide, goserelin, and a combination of any two or more thereof.
AP. The method of any one of Paragraphs AD-AO, wherein the administering comprises oral administration, intravenous administration, or intramuscular administration.
AQ. The method of any one of Paragraphs AD-AP, wherein the method comprises orally administering to the subject the effective amount of the compound.
AR. The method of any one of Paragraphs AD-AQ, wherein the administering comprises injection of the compound into the neoplasm or proximal to the neoplasm.

Other embodiments are set forth in the following claims, along with the full scope of equivalents to which such claims are entitled.

1. A method of treating a cancer in a subject, the method comprising administering to the subject an effective amount of a compound to treat the cancer; wherein the compound is at least one of darapladib, rilapladib, AA39-2, or ML256; and wherein the cancer harbors a constitutively active variant of one or both of KRAS or HRAS, wherein the constitutively active variant is a gain of function mutation, a duplication, or a gene amplification; optionally wherein the cancer exhibits elevated expression and/or activity of at least one of PLA2G7A and PAFAH2 compared to healthy control cells.

2. The method of claim 1, wherein the cancer is a pancreatic cancer, a colorectal cancer, a hepatocellular cancer, a bile duct cancer, a soft tissue sarcoma, a blood or hematopoietic cell cancer, a breast cancer, a lung cancer, a uterine or cervical cancer, a thyroid cancer, a bladder cancer, a kidney cancer, a gastric cancer, an ovarian cancer, a brain cancer, a mesothelioma cancer, a skin cancer, a head and neck cancer, a neuroendocrine cancer or neoplasm, an esophagus cancer, a testicular cancer, a prostate cancer, or a thymus cancer.

3. The method of claim 1, wherein the cancer comprises an adenoma, an adenocarcinoma, a uterine carcinoma, a squamous cell carcinoma, a small cell carcinoma, a transitional carcinoma, a serous carcinoma, a clear-cell carcinoma, a mucinous adenocarcinoma, an undifferentiated carcinoma, a dedifferentiated carcinoma, a serous adenocarcinoma, a sarcoma, a myeloma, a leukemia, a lymphoma, a dysplastic lesion, or a combination of any two or more thereof.

4. The method of claim 1, wherein the subject is human.

5.-10. (canceled)

11. The method of claim 1, wherein the administering further comprises administration of a chemotherapeutic agent selected from the group consisting of an alkylating agent; a nitrosourea; an antimetabolite; an anthracycline; a topoisomerase II inhibitor; a mitotic inhibitor; an anti-estrogen; a progestin; an aromatase inhibitor; an anti-androgen; an LHRH agonist; a corticosteroid hormone; a DNA alkylating agent; a taxane; a vinca alkaloid; a microtubule poison, and a combination of any two or more thereof.

12.-13. (canceled)

14. The method of claim 1, wherein the method comprises orally administering to the subject the effective amount of the compound to treat the cancer.

15. A method of slowing or reversing growth of a tumor in a subject, the method comprising administering to the subject an effective amount of a compound; wherein the compound is at least one of darapladib, rilapladib, AA39-2, or ML256; wherein the effective amount is an amount effective to slow or reverse growth of the tumor; and wherein the tumor is of a cancer that harbors a constitutively active variant of one or both of KRAS or HRAS, wherein the constitutively active variant is a gain of function mutation, a duplication, or a gene amplification; optionally wherein the cancer exhibits elevated expression and/or activity of at least one of PLA2G7A and PAFAH2 compared to healthy control cells.

16. The method of claim 15, wherein the tumor is of a cancer selected from a pancreatic cancer, a colorectal cancer, a hepatocellular cancer, a bile duct cancer, a soft tissue sarcoma, a blood or hematopoietic cell cancer, a breast cancer, a lung cancer, a uterine or cervical cancer, a thyroid cancer, a bladder cancer, a kidney cancer, a gastric cancer, an ovarian cancer, a brain cancer, a mesothelioma cancer, a skin cancer, a head and neck cancer, a neuroendocrine cancer or neoplasm, an esophagus cancer, a testicular cancer, a prostate cancer, or a thymus cancer.

17. The method of claim 15, wherein the tumor is of a cancer comprising an adenoma, an adenocarcinoma, a uterine carcinoma, a squamous cell carcinoma, a small cell carcinoma, a transitional carcinoma, a serous carcinoma, a clear-cell carcinoma, a mucinous adenocarcinoma, an undifferentiated carcinoma, a dedifferentiated carcinoma, a serous adenocarcinoma, a sarcoma, a myeloma, a leukemia, a lymphoma, a dysplastic lesion, or a combination of any two or more thereof.

18. The method of claim 15, wherein the subject is human.

19.-24. (canceled)

25. The method of claim 15, wherein the administering further comprises administration of a chemotherapeutic agent selected from the group consisting of an alkylating agent; a nitrosourea; an antimetabolite; an anthracycline; a topoisomerase II inhibitor; a mitotic inhibitor; an anti-estrogen; a progestin; an aromatase inhibitor; an anti-androgen; an LHRH agonist; a corticosteroid hormone; a DNA alkylating agent; a taxane; a vinca alkaloid; a microtubule poison, and a combination of any two or more thereof.

26. (canceled)

27. The method of claim 15, wherein the administering comprises oral administration, intravenous administration, or intramuscular administration.

28. (canceled)

29. The method of claim 15, wherein the administering comprises injection of the compound into the tumor or proximal to the tumor.

30. A method of slowing or reversing growth of a neoplasm in a subject and/or slowing or reversing proliferation of the neoplasm in the subject, the method comprising administering to the subject an effective amount of a compound; where the compound is at least one of darapladib, rilapladib, AA39-2, or ML256; wherein the effective amount is an amount effective to slow or reverse growth of the neoplasm and/or slow or reverse proliferation of the neoplasm; and wherein the neoplasm is of a cancer that harbors a constitutively active variant of one or both of KRAS or HRAS, wherein the constitutively active variant is a gain of function mutation, a duplication, or a gene amplification; optionally wherein the cancer exhibits elevated expression and/or activity of at least one of PLA2G7A and PAFAH2 compared to healthy control cells.

31. The method of claim 30, wherein the neoplasm is of a cancer selected from a pancreatic cancer, a colorectal cancer, a hepatocellular cancer, a bile duct cancer, a soft tissue sarcoma, a blood or hematopoietic cell cancer, a breast cancer, a lung cancer, a uterine or cervical cancer, a thyroid cancer, a bladder cancer, a kidney cancer, a gastric cancer, an ovarian cancer, a brain cancer, a mesothelioma cancer, a skin cancer, a head and neck cancer, a neuroendocrine cancer or neoplasm, an esophagus cancer, a testicular cancer, a prostate cancer, or a thymus cancer.

32. The method of claim 30, wherein the neoplasm is of a cancer comprising an adenoma, an adenocarcinoma, a uterine carcinoma, a squamous cell carcinoma, a small cell carcinoma, a transitional carcinoma, a serous carcinoma, a clear-cell carcinoma, a mucinous adenocarcinoma, an undifferentiated carcinoma, a dedifferentiated carcinoma, a serous adenocarcinoma, a sarcoma, a myeloma, a leukemia, a lymphoma, a dysplastic lesion, or a combination of any two or more thereof.

33. The method of claim 30, wherein the subject is human.

34.-39. (canceled)

40. The method of claim 30, wherein the administering further comprises administration of a chemotherapeutic agent selected from the group consisting of an alkylating agent; a nitrosourea; an antimetabolite; an anthracycline; a topoisomerase II inhibitor; a mitotic inhibitor; an anti-estrogen; a progestin; an aromatase inhibitor; an anti-androgen; an LHRH agonist; a corticosteroid hormone; a DNA alkylating agent; a taxane; a vinca alkaloid; a microtubule poison, and a combination of any two or more thereof.

41. (canceled)

42. The method of claim 30, wherein the administering comprises oral administration, intravenous administration, or intramuscular administration.

43. (canceled)

44. The method of claim 30, wherein the administering comprises injection of the compound into the neoplasm or proximal to the neoplasm.
